(19) United States
(12) Patent Application Publication
(10) Pub. No.: US 2025/0314664 A1
(43) Pub. Date: Oct. 9, 2025

(54) COMPOSITIONS AND METHODS TO DETECT INFECTIOUS ORGANISMS

(71) Applicant: Mars, Incorporated, McLean, VA (US)

(72) Inventors: Christian Leutenegger, Dixon, CA (US); Irina Sokolchik, Whitestown, IN (US); Isabelle Caya, Drummondville (CA)

(73) Assignee: Mars, Incorporated, McLean, VA (US)

(21) Appl. No.: 19/095,281

(22) Filed: Mar. 31, 2025

Related U.S. Application Data
(60) Provisional application No. 63/573,691, filed on Apr. 3, 2024.

(51) Int. Cl. G01N 33/68; A61K 31/65; A61P 31/04; C07K 1/14; C07K 14/00; C12N 15/62; G01N 33/569

(52) U.S. Cl. CPC G01N 33/6893; A61K 31/65; A61P 31/04; C07K 1/14; C07K 14/00; C12N 15/62; G01N 33/56911; C07K 2319/00; G01N 2800/26

(57) ABSTRACT

The present disclosure relates to compositions and methods for the detection of infectious organisms, including Ehrlichia spp. In certain embodiments, the infectious organism is Ehrlichia ewingii.

### CROSS-REFERENCES TO RELATED APPLICATIONS

This application claims the benefit of priority to U.S. Provisional Patent Application No. 63/573,691, filed Apr. 3, 2024, the content of which is incorporated herein by reference in its entirety, and to which priority is claimed.

### SEQUENCE LISTING

The instant application contains a Sequence Listing which has been submitted via Patent Center and is hereby incorporated by reference in its entirety. Said .xml copy, created on Mar. 31, 2025, is named 0692690735, and is 27,629 bytes in size.

### FIELD

The present disclosure relates to compositions and methods for the detection of infectious organisms, including Ehrlichia spp. In certain embodiments, the infectious organism is Ehrlichia ewingii.

### BACKGROUND

The Ehrlichiae are a group of gram-negative, obligate intracellular cocci that infect different blood cells in many animal species, as well as in humans. Although these pathogens share phylogenetic similarities, there are multiple differences from tick vectors to host-cell tropism and clinical manifestations. Dogs are susceptible to infection with multiple Ehrlichia spp.; among those are E. canis, transmitted by Rhipicephalus sanguineus (brown dog tick), and E. chaffeensis and E. ewingii, whose primary vector is Amblyomma americanum (lone star tick) (Beall M J, 2012). Coinfections with multiple ehrlichial agents have been reported in dogs (Kordick, 1999). The lone star tick is widely distributed in the eastern, southeastern, and south-central United States, and the brown dog tick is found worldwide (CDC.gov).

In dogs, two leukotrophic diseases are caused by bacteria in the genus Ehrlichia: Canine Monocytic Ehrlichiosis (CME) mainly caused by E. canis and E. chaffeensis, and Canine Granulocytic Ehrlichiosis (CGE), caused by E. ewingii (Aziz, 2023). In recent decades, enhanced and modified transmission patterns of all vector-borne pathogens have been recorded around the globe (Dantas-Torres, 2015) due to multiple factors, including an increase in human population, deforestation, as well as frequent transport of pet animals from one continent to another.

A variety of serologic tests, such as indirect fluorescent antibody (IFA), Western Blot, and ELISAs have been used to verify past or current infections. IFA is the reference standard serologic test for diagnosis of Ehrlichiosis (CDC.gov) and is being widely used testing method due to its simplicity and cost-effectiveness compared to other approaches.

However, shortcomings of the IFA test are antigenic cross-reactivity with other closely related species that infect dogs, the nature of antigens used in the tests (Stillman B A, 2014), as well as subjective endpoint interpretations and lack of standardization between laboratories (Luo, T., 2010). Since E. ewingii has not been yet cultured, IFAs for antibodies against other Ehrlichia spp. have been used for anti-E. ewingii antibodies detection (Yabsley M J, 2011). Examination of a peripheral blood smear to identify thrombocytopenia and to visually inspect cells for morula is often the only diagnostic test performed prior to treatment (Cohn, L A, 2012).

Identification and differentiation of Ehrlichia and related species with PCR testing is useful in dogs with subclinical & persistent infections, yet some dogs might be PCR negative if the organism is sequestered or persists at undetectable levels, and not so helpful after antibiotic therapy is initiated (Luo, T, 2010). According to the CDC, “a negative PCR result does not rule out the diagnosis, and treatment should not be withheld due to a negative result” (CDC.gov).

Thus, there is a need for novel sensitive and reliable diagnostic tools.

### SUMMARY OF THE INVENTION

In certain non-limiting embodiments, the present disclosure provides a fusion protein comprising the amino sequence set forth in SEQ ID NO: 1 or a variant thereof, SEQ ID NO: 2 or a variant thereof, SEQ ID NO: 3 or a variant thereof, SEQ ID NO: 4 or a variant thereof, SEQ ID NO: 5 or a variant thereof, SEQ ID NO: 6 or a variant thereof, SEQ ID NO: 7 or a variant thereof, SEQ ID NO: 8 or a variant thereof, SEQ ID NO: 9 or a variant thereof, or a combination thereof. In certain non-limiting embodiments, the present disclosure also provides a fusion protein comprising the amino sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof.

In certain non-limiting embodiments, the present disclosure provides a fusion protein comprising at least two polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein comprises at least five polypeptides. In certain embodiments, the fusion protein comprises at least eight polypeptides. In certain embodiments, the fusion protein comprises at least eleven polypeptides.

In certain embodiments, the fusion protein further comprises a linker. In certain embodiments, the fusion protein has an isoelectric point between about 4.50 and about 4.60. In certain embodiments, the isoelectric point is about 4.58. In certain embodiments, the fusion protein has a molecular weight between about 23.0 kDa and about 27.0 kDa. In certain embodiments, the molecular weight is about 24.3 kDa, 24.4 kDa, or 24.8 kDa. In certain embodiments, the molecular weight is about 24.8 kDa.

In certain embodiments, the fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 10, SEQ ID NO: 11, SEQ ID NO: 12, SEQ ID NO: 13, SEQ ID NO: 14, or SEQ ID NO: 15. In certain embodiments, the fusion protein comprises the amino acid sequence set forth in SEQ ID NO: 10, SEQ ID NO: 11, SEQ ID NO: 12, SEQ ID NO: 13, SEQ ID NO: 14, or SEQ ID NO: 15. In certain embodiments, the fusion protein consists of the amino acid sequence set forth in SEQ ID NO: 10, SEQ ID NO: 11, SEQ ID NO: 12, SEQ ID NO: 13, SEQ ID NO: 14, or SEQ ID NO: 15.

In certain embodiments, the fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 12. In certain embodiments, the fusion protein comprises the amino acid sequence set forth in SEQ ID NO: 12. In certain embodiments, the fusion protein consists of the amino acid sequence set forth in SEQ ID NO: 12.

In certain embodiments, the fusion protein further comprises a tag.

In certain non-limiting embodiments, the present disclosure provides a nucleic acid molecule comprising a polynucleotide encoding the fusion protein disclosed herein. Moreover, the present disclosure provides a vector comprising the nucleic acid molecule disclosed herein. Also provided is a host cell comprising the fusion protein, the nucleic acid molecule, or the vector disclosed herein. In certain embodiments, the host cell is E. coli. In certain non-limiting embodiments, the present disclosure provides a method of producing a fusion protein comprising culturing the host cell disclosed herein in a culture medium and purifying the fusion protein.

In certain non-limiting embodiments, the present disclosure provides a composition or a solid phase support comprising a fusion protein disclosed herein. In certain embodiments, the composition or the solid phase support further comprises a second fusion protein. In certain embodiments, the second fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 16 or SEQ ID NO: 17. In certain embodiments, the second fusion protein comprises the amino acid sequence set forth in SEQ ID NO: 16 or SEQ ID NO: 17. In certain embodiments, the second fusion protein consists of the amino acid sequence set forth in SEQ ID NO: 16 or SEQ ID NO: 17. In certain embodiments, the composition or the solid phase support further comprises a second fusion protein and a third fusion protein. In certain embodiments, a) the second fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 16, and b) the third fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 17. In certain embodiments, a) the second fusion protein comprises the amino acid sequence set forth in SEQ ID NO: 16, and b) the third fusion protein comprises the amino acid sequence set forth in SEQ ID NO: 17. In certain embodiments, a) the second fusion protein consists of the amino acid sequence set forth in SEQ ID NO: 16, and b) the third fusion protein consists of the amino acid sequence set forth in SEQ ID NO: 17.

In certain non-limiting embodiments, the present disclosure provides a method of detecting an antibody binding an infectious organism in a subject comprising contacting the fusion protein, the composition, or the solid phase support disclosed herein with a sample from the subject and detecting a complex formed between the antibody and the fusion protein. In certain embodiments, the infectious organism is E. ewingii. In certain embodiments, the antibody specifically binds to an epitope of E. ewingii. In certain embodiments, the sample is a serum sample, a plasma sample, a blood sample, or a combination thereof. In certain embodiments, the fusion protein is immobilized on a solid phase support. In certain embodiments, the subject is a dog.

In certain non-limiting embodiments, the present disclosure provides a method of identifying a subject having an E. ewingii infection. In certain embodiments, the method comprises contacting the fusion protein, the composition, or the solid phase support disclosed herein with a sample from the subject. In certain embodiments, the method comprises detecting a complex formed between the sample and the fusion protein, the composition, or the solid phase. In certain embodiments, an increased level of detected complexes relative to a normal control indicates that the subject has an E. ewingii infection. In certain embodiments, the sample is a serum sample, a plasma sample, a blood sample, or a combination thereof. In certain embodiments, the subject is a dog.

In certain non-limiting embodiments, the present disclosure provides a method of identifying a subject having Lyme disease. In certain embodiments, the method comprises contacting the fusion protein, the composition, or the solid phase support disclosed herein with a sample from the subject. In certain embodiments, the method comprises detecting a complex formed between the sample and the fusion protein, the composition, or the solid phase. In certain embodiments, an increased level of detected complexes relative to a normal control indicates that the subject has Lyme disease. In certain embodiments, the sample is a serum sample, a plasma sample, a blood sample, or a combination thereof. In certain embodiments, the subject is a dog.

In certain non-limiting embodiments, the present disclosure provides a kit comprising contacting the fusion protein, the composition, or the solid phase support disclosed herein.

In certain non-limiting embodiments, the present disclosure provides a method of preventing, treating, and/or delaying progression of E. ewingii infection of a subject in need thereof comprising administering an antibiotic to the subject. In certain embodiments, the antibiotic is selected from clindamycin, metronidazole, amoxicillin, cephalexin, doxycycline, enrofloxacin, gentamicin, trimethoprim, sulfamethoxazole, clavamox, chloramphenicol, cefpodoxime, tetracycline, marbofloxacin, antirobe, ciprofloxacin, albon, amoxicillin, baytril, biomox amoxicillin, cephalexin, amoxicillin, clavulanic acid, ketoconazole, sulfadimethoxine, or a combination thereof. In certain embodiments, the antibiotic is doxycycline. In certain embodiments, the subject is a dog.

In certain non-limiting embodiments, the present disclosure provides a method of treating and/or delaying progression of E. ewingii infection of a subject in need thereof. In certain embodiments, the method comprises identifying the subject as having an E. ewingii infection, and administering an antibiotic to the subject. In certain embodiments, the identifying comprises contacting the fusion protein, the composition, or the solid phase support disclosed herein with a sample from the subject. In certain embodiments, the identifying comprises detecting a complex formed between the sample and the fusion protein, the composition, or the solid phase. In certain embodiments, an increased level of detected complexes relative to a normal control indicates that the subject has an E. ewingii infection. In certain embodiments, the antibiotic is selected from clindamycin, metronidazole, amoxicillin, cephalexin, doxycycline, enrofloxacin, gentamicin, trimethoprim, sulfamethoxazole, clavamox, chloramphenicol, cefpodoxime, tetracycline, marbofloxacin, antirobe, ciprofloxacin, albon, amoxicillin, baytril, biomox amoxicillin, cephalexin, amoxicillin, clavulanic acid, ketoconazole, sulfadimethoxine, or a combination thereof. In certain embodiments, the antibiotic is doxycycline. In certain embodiments, the subject is a dog.

### DETAILED DESCRIPTION

To date, there is a need to identify and differentiate Ehrlichia spp. since false negatives are often observed using conventional methods (e.g., qPCR). The present disclosure relates to compositions and methods that can be used to improve the detection of infectious diseases. For purposes of clarity of disclosure and not by way of limitation, the detailed description is divided into the following subsections:
1. Definitions;
2. Fusion Proteins;
3. Methods of Detection;
4. Computer Readable Medium;
5. Kits; and
6. Methods of Treatment.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “nucleic acid molecule” and “nucleotide sequence,” as used herein, refers to a single or double-stranded covalently linked sequence of nucleotides in which phosphodiester bonds join the 3′ and 5′ ends on each nucleotide. The nucleic acid molecule can include deoxyribonucleotide bases or ribonucleotide bases and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residues is an artificial chemical mimetic of a corresponding naturally occurring amino acid, as well as to naturally occurring amino acid polymers and non-naturally occurring amino acid polymers.

The term “amino acid,” as used herein, can be naturally occurring and synthetic amino acids, as well as amino acid analogs and amino acid mimetics that function like the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs and derivatives can refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, and methionine methyl sulfonium. Such analogs can have modified R groups (e.g., norleucine) or modified peptide backbones, but retain the same basic chemical structure as a naturally occurring amino acid. Amino acid mimetics means chemical compounds with a structure that is different from the general chemical structure of an amino acid, but that function similarly to a naturally occurring amino acid. Non-limiting examples of amino acids include tryptophan, phenylalanine, histidine, glycine, cysteine, alanine, tyrosine, serine, methionine, asparagine, leucine, asparagine, threonine, isoleucine, proline, glutamic acid, aspartic acid, hydroxyl proline, arginine, cystine, glutamine, lysine, valine, ornithine, taurine, and combinations thereof.

As used herein, the term “antibody” refers to an immunoglobulin molecule, or a fragment thereof (e.g., Fab, Fab′, F(ab′)2, Fv, single chain (scFv)), that specifically binds to a target (e.g., an antigen, a carbohydrate, polynucleotide, lipid, polypeptide, etc.), through at least one antigen recognition site, located in the variable region of the immunoglobulin molecule. An antibody can be an immunoglobulin of any class including, for example, and without any limitation, IgG, IgM, IgA, IgD, IgE, and IgY. As used herein, an antibody can be a polyclonal antibody or a monoclonal antibody. In certain embodiments, the term antibody also refers to mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a peptide sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence. There is no critical upper limit to the length of the fragment, which may, for example, comprise nearly the full length of the antigen sequence, or even a fusion protein comprising two or more epitopes from the target antigen. An epitope for use in the subject invention is not limited to a peptide having the exact sequence of the portion of the parent protein from which it is derived but also encompasses sequences identical to the native sequence, as well as modifications to the native sequence, such as deletions, additions, and substitutions (conservative).

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include but are not limited to, body fluids, such as blood, plasma, serum, cerebrospinal fluid, synovial fluid, urine and sweat, tissue and organ samples from animals and plants, and processed samples derived therefrom. Also included are soil and water samples and other environmental samples, viruses, bacteria, fungi, algae, protozoa, and components thereof.

The terms “level” or “levels” are used to refer to the presence and/or amount of protein, and can be determined qualitatively or quantitatively. A “qualitative” change in the protein level refers to the appearance or disappearance of a protein spot that is not detectable or is present in samples obtained from normal controls. A “quantitative” change in the levels of one or more proteins of the profile refers to a measurable increase or decrease in the protein levels when compared to a healthy control.

A “healthy control” or “normal control” is a biological sample taken from an individual who does not suffer from an infectious disorder. A “negative control,” is a sample that lacks any of the specific analyte the assay is designed to detect and thus provides a reference baseline for the assay.

The term “isolated,” as used herein, refers to a material that is removed from at least one component with which it is naturally associated (e.g., removed from its original environment).

As used herein, the terms “reduce” and “reduction” refer to a measurable lessening of an endpoint (e.g., enzymatic activity, production of the compound, expression of a protein) by at least about 10%, at least about 50%, at least about 75%, or at least about 90%. In certain embodiments, the reduction can be from about 10% to about 100%.

As used herein, the term “increase,” “elevate” and “elevation” refers to a measurable augmentation of an endpoint (e.g., enzymatic activity, production of compound, expression of a protein) by at least about 10%, at least about 50%, at least about 75%, or at least about 90%. In certain embodiments, the increase can be from about 10% to about 100%. In certain embodiments, the increase can be at least about 10-fold, about 100-fold, or about 1000-fold or more. In certain embodiments, the increase can be about 100-fold or more, about 1000-fold or more, or about 10,000-fold or more.

As used herein, “vector (or plasmid)” refers to discrete elements that are used to introduce heterologous DNA into cells for either expression or replication thereof. Selection and use of such vehicles are well-known within the skill of the artisan. An expression vector includes vectors capable of expressing DNA that are operatively linked with regulatory sequences, such as promoter regions, that are capable of affecting the expression of such DNA fragments. Thus, an expression vector refers to a recombinant DNA or RNA construct, such as a plasmid, a phage, a recombinant virus, or another vector that, upon introduction into an appropriate host cell, results in the expression of the cloned DNA. Appropriate expression vectors are well known to those of skill in the art and include those that are replicable in eukaryotic cells and/or prokaryotic cells and those that remain episomal or those that integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding, and transcription initiation. This portion of the promoter region is referred to as the promoter. In addition, the promoter region includes sequences that modulate this recognition, binding, and transcription initiation activity of RNA polymerase. These sequences may be cis-acting or may be responsive to trans-acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked or operationally associated” refers to the functional relationship of DNA with regulatory and effector sequences of nucleotides, such as promoters, enhancers, transcriptional and translational stop sites, and other signal sequences. For example, the operative linkage of DNA to a promoter refers to the physical and functional relationship between the DNA and the promoter such that the transcription of such DNA is initiated from the promoter by an RNA polymerase that specifically recognizes, binds to, and transcribes the DNA. To improve expression and/or in vitro transcription, it may be necessary to remove, add, or alter 5′ untranslated portions of the clones to eliminate extra, potentially inappropriate alternative translation initiation (i.e., start) codons or other sequences that may interfere with or reduce expression, either at the level of transcription or translation. Alternatively, consensus ribosome binding sites can be inserted immediately 5′ of the start codon and may enhance expression. See, e.g., Kozak (1991) J. Biol. Chem. 266:19867-19870. The desirability of (or need for) such modification may be empirically determined.

### 2. Fusion Proteins

In certain embodiments, the present disclosure provides compositions and methods for the detection of Ehrlichia spp. Ehrlichiae are obligate intracellular bacteria that grow within membrane-bound vacuoles in human and animal leukocytes. The two most important species to infect humans include E. chaffeensis, the causative agent of human monocytic ehrlichiosis (HME), and A. phagocytophilum, the agent of human granulocytic anaplasmosis (HGA). Less commonly, ehrlichiosis is caused by Ehrlichia ewingii, which was discovered in 1999.

In 2009, a third species of Ehrlichia was identified in four patients from Wisconsin and Minnesota who had fever, malaise, headache, and lymphopenia; more than 100 cases have been subsequently reported. Molecular methods, culture techniques, and serologic testing demonstrated that this species is closely related to Ehrlichia muris, which is found in Eastern Europe and Asia. In 2007, this organism was named E. muris eauclairensis.

### 2.1. Fusion Proteins

In certain embodiments, the present disclosure provides a fusion protein that can be used for the detection of Ehrlichiae spp. Additionally, the present disclosure provides a composition comprising the presently disclosed fusion proteins. In certain embodiments, the presently disclosed fusion protein includes a combination of peptides that can be recognized by antibodies targeting Ehrlichiae spp.

In certain embodiments, the fusion protein comprises the amino acid sequence set forth in SEQ ID NO: 1 or a variant thereof. In certain embodiments, the fusion protein comprises the amino acid sequence set forth in SEQ ID NO: 1. In certain embodiments, the fusion protein comprises the amino acid sequence set forth in SEQ ID NO: 2 or a variant thereof. In certain embodiments, the fusion protein comprises the amino acid sequence set forth in SEQ ID NO: 2. In certain embodiments, the fusion protein comprises the amino acid sequence set forth in SEQ ID NO: 3 or a variant thereof. In certain embodiments, the fusion protein comprises the amino acid sequence set forth in SEQ ID NO: 3. In certain embodiments, the fusion protein comprises the amino acid sequence set forth in SEQ ID NO: 4 or a variant thereof. In certain embodiments, the fusion protein comprises the amino acid sequence set forth in SEQ ID NO: 4. In certain embodiments, the fusion protein comprises the amino acid sequence set forth in SEQ ID NO: 5 or a variant thereof. In certain embodiments, the fusion protein comprises the amino acid sequence set forth in SEQ ID NO: 5. In certain embodiments, the fusion protein comprises the amino acid sequence set forth in SEQ ID NO: 6 or a variant thereof. In certain embodiments, the fusion protein comprises the amino acid sequence set forth in SEQ ID NO: 6. In certain embodiments, the fusion protein comprises the amino acid sequence set forth in SEQ ID NO: 7 or a variant thereof. In certain embodiments, the fusion protein comprises the amino acid sequence set forth in SEQ ID NO: 7. In certain embodiments, the fusion protein comprises the amino acid sequence set forth in SEQ ID NO: 8 or a variant thereof. In certain embodiments, the fusion protein comprises the amino acid sequence set forth in SEQ ID NO: 8. In certain embodiments, the fusion protein comprises the amino acid sequence set forth in SEQ ID NO: 9 or a variant thereof. In certain embodiments, the fusion protein comprises the amino acid sequence set forth in SEQ ID NO: 9.

In certain embodiments, the fusion protein includes at least one polypeptide comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least two polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least three polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least four polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least five polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least six polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least seven polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least eight polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least nine polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least ten polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least eleven polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least twelve polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least thirteen polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least fourteen polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. In certain embodiments, the fusion protein includes at least fifteen polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof. SEQ ID NOs: 1-9 are provided below.

SEQ ID NO: 1
AETKKTFGLEVSYDGAKIED
SEQ ID NO: 2
AETIATFGLSKTYNGAQITD
SEQ ID NO: 3
AETKKVLGLNKNYDGAKIED
SEQ ID NO: 4
AETKRTFGLDKNEGLTD
SEQ ID NO: 5
AETKNSIALEKNYDGAKIED
SEQ ID NO: 6
AETSKKFVLENNYDGAKIED
SEQ ID NO: 7
AETKRTFGLDIGYFGAKIED
SEQ ID NO: 8
AETKKTFGLESFYEGAKIED
SEQ ID NO: 9
AETKKTFGLEGSYEGAKIED

In certain embodiments, the fusion protein comprises one or more copies of the amino acid sequence set forth in SEQ ID NO: 1 or a variant thereof. In certain embodiments, the fusion protein comprises one or more copies of the amino acid sequence set forth in SEQ ID NO: 1. In certain embodiments, the fusion protein comprises one or more copies of the amino acid sequence set forth in SEQ ID NO: 2 or a variant thereof. In certain embodiments, the fusion protein comprises one or more copies of the amino acid sequence set forth in SEQ ID NO: 2. In certain embodiments, the fusion protein comprises one or more copies of the amino acid sequence set forth in SEQ ID NO: 3 or a variant thereof. In certain embodiments, the fusion protein comprises one or more copies of the amino acid sequence set forth in SEQ ID NO: 3. In certain embodiments, the fusion protein comprises one or more copies of the amino acid sequence set forth in SEQ ID NO: 4 or a variant thereof. In certain embodiments, the fusion protein comprises one or more copies of the amino acid sequence set forth in SEQ ID NO: 4. In certain embodiments, the fusion protein comprises one or more copies of the amino acid sequence set forth in SEQ ID NO: 5 or a variant thereof. In certain embodiments, the fusion protein comprises one or more copies of the amino acid sequence set forth in SEQ ID NO: 5. In certain embodiments, the fusion protein comprises one or more copies of the amino acid sequence set forth in SEQ ID NO: 6 or a variant thereof. In certain embodiments, the fusion protein comprises one or more copies of the amino acid sequence set forth in SEQ ID NO: 6. In certain embodiments, the fusion protein comprises one or more copies of the amino acid sequence set forth in SEQ ID NO: 7 or a variant thereof. In certain embodiments, the fusion protein comprises one or more copies of the amino acid sequence set forth in SEQ ID NO: 7. In certain embodiments, the fusion protein comprises one or more copies of the amino acid sequence set forth in SEQ ID NO: 8 or a variant thereof. In certain embodiments, the fusion protein comprises one or more copies of the amino acid sequence set forth in SEQ ID NO: 8. In certain embodiments, the fusion protein comprises one or more copies of the amino acid sequence set forth in SEQ ID NO: 9 or a variant thereof. In certain embodiments, the fusion protein comprises one or more copies of the amino acid sequence set forth in SEQ ID NO: 9.

In certain embodiments, the fusion protein comprises a tag sequence. As used herein, a “tag” or an “epitope tag” refers to a sequence of amino acids, typically added to the N- and/or C-terminus of a polypeptide. The inclusion of tags fused to a polypeptide can facilitate polypeptide purification and/or detection. In certain embodiments, a tag or tag polypeptide refers to a polypeptide that has enough residues to provide an epitope recognized by an antibody or can serve for detection or purification, yet is short enough such that it does not interfere with the activity of chimeric polypeptide to which it is linked. The tag polypeptide typically is sufficiently unique so an antibody that specifically binds thereto does not substantially cross-react with epitopes in the polypeptide to which it is linked. Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well-known and can be readily synthesized and designed. Exemplary tag polypeptides include those used for affinity purification and include His tags, the influenza hemagglutinin (HA) tag polypeptide, and its antibody 12CA5; the c-Myc tag and the 8F9, 3C7, 6E10, G4, B7 and 9E10 antibodies thereto; and the Herpes Simplex virus glycoprotein D (gD) tag and its antibody. See, e.g., Field et al. (1988) Mol. Cell. Biol. 8:2159-2165; Evan et al. (1985) Mol. Cell. Biol. 5:3610-3616; Paborsky et al. (1990) Protein Engineering 3:547-553.

In certain embodiments, the fusion protein comprises an amino acid linker between the polypeptides. Linkers are short polypeptide sequences, typically between 1 amino acid residue and 20 amino acid residues, that occur between polypeptides (e.g., one having an amino acid sequence set forth in SEQ ID Nos: 1-9). Linkers are often composed of flexible residues like glycine and serine so that the adjacent polypeptides are free to move relative to one another. Conventionally, longer linkers are used when it is necessary to ensure that two adjacent domains do not sterically interfere with one another.

In certain embodiments, the presently disclosed fusion protein has an isoelectric point between about 4.50 and about 4.60. In certain embodiments, the presently disclosed fusion protein has an isoelectric point between about 4.51 and about 4.60, between about 4.52 and about 4.60, between about 4.53 and about 4.60, between about 4.54 and about 4.60, between about 4.55 and about 4.60, between about 4.56 and about 4.60, between about 4.57 and about 4.60, between about 4.58 and about 4.60, between about 4.59 and about 4.60, between about 4.50 and about 4.59, between about 4.50 and about 4.58, between about 4.50 and about 4.57, between about 4.50 and about 4.56, between about 4.50 and about 4.55, between about 4.50 and about 4.54, between about 4.50 and about 4.53, between about 4.50 and about 4.52, between about 4.50 and about 4.51, between about 4.52 and about 4.54, between about 4.55 and about 4.57, or between about 4.57 and about 4.59. In certain embodiments, the presently disclosed fusion protein has an isoelectric point of about 4.58.

In certain embodiments, the presently disclosed fusion protein has a molecular weight between about 23.0 kDa and about 27.0 kDa. In certain embodiments, the presently disclosed fusion protein has a molecular weight between about 23.0 kDa and about 27.0 kDa, between about 23.0 kDa and about 26.5 kDa, between about 23.0 kDa and about 25.0 kDa, between about 23.0 kDa and about 25.5 kDa, between about 23.0 kDa and about 25.0 kDa, between about 23.0 kDa and about 24.5 kDa, between about 23.0 kDa and about 24.0 kDa, between about 24.0 kDa and about 24.1 kDa, between about 24.0 kDa and about 24.2 kDa, between about 24.0 kDa and about 24.3 kDa, between about 24.0 kDa and about 24.4 kDa, between about 24.0 kDa and about 24.5 kDa, between about 24.0 kDa and about 24.6 kDa, between about 24.0 kDa and about 24.7 kDa, between about 24.0 kDa and about 24.8 kDa, between about 24.0 kDa and about 24.9 kDa, between about 24.2 kDa and about 24.5 kDa, between about 24.2 kDa and about 24.3 kDa, between about 24.2 kDa and about 24.4 kDa, between about 24.3 kDa and about 24.4 kDa, between about 24.3 kDa and about 24.5 kDa, between about 24.7 kDa and about 24.8 kDa, between about 24.8 kDa and about 24.9 kDa, or between about 24.7 kDa and about 24.9 kDa. In certain embodiments, the presently disclosed fusion protein has a molecular weight of about 24.3 kDa. In certain embodiments, the presently disclosed fusion protein has a molecular weight of about 24.4 kDa. In certain embodiments, the presently disclosed fusion protein has a molecular weight of about 24.8 kDa.

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 10. In certain embodiments, the presently disclosed fusion protein comprises the amino acid sequence set forth in SEQ ID NO: 10. In certain embodiments, the presently disclosed fusion protein consists of the amino acid sequence set forth in SEQ ID NO: 10. SEQ ID NO: 10 is provided below.

[SEQ ID NO: 10]
AETKKTFGLEVSYDGAKIEDGGAETIATFGLSKTYNGAQITDGGA
ETKKVLGLNKNYDGAKIEDGAETKRTFGLDKNEGLTDGGAETKNS
IALEKNYDGAKIEDGGAETIATFGLSKTYNGAQITDAETSKKFVL
ENNYDGAKIEDGGAETKRTFGLDIGYFGAKIEDGAETKKTFGLES
FYEGAKIEDAETKKTFGLEGSYEGAKIEDGAETIATFGLSKTYNG
AQITD

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 11. In certain embodiments, the presently disclosed fusion protein comprises the amino acid sequence set forth in SEQ ID NO: 11. In certain embodiments, the presently disclosed fusion protein consists of the amino acid sequence set forth in SEQ ID NO: 11. SEQ ID NO: 11 is provided below.

[SEQ ID NO: 11]
AETKKTFGLEVSYDGAKIEDGGAETIATFGLSKTYNGAQITDGGA
ETKKVLGLNKNYDGAKIEDGAETKRTFGLDKNEGLTDGGAETKNS
IALEKNYDGAKIEDGGAETIATFGLSKTYNGAQITDGAETSKKEV
LENNYDGAKIEDGGAETKRTFGLDIGYFGAKIEDGAETKKTFGLE
SFYEGAKIEDGAETKKTFGLEGSYEGAKIEDGAETIATFGLSKTY
NGAQITD

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 12. In certain embodiments, the presently disclosed fusion protein comprises the amino acid sequence set forth in SEQ ID NO: 12. In certain embodiments, the presently disclosed fusion protein consists of the amino acid sequence set forth in SEQ ID NO: 12. SEQ ID NO: 12 is provided below.

[SEQ ID NO: 12]
SNIGAETKKTFGLEVSYDGAKIEDGGAETIATFGLSKTYNGAQIT
DGGAETKKVLGLNKNYDGAKIEDGAETKRTFGLDKNEGLTDGGAE
TKNSIALEKNYDGAKIEDGGAETIATFGLSKTYNGAQITDGAETS
KKFVLENNYDGAKIEDGGAETKRTFGLDIGYFGAKIEDGAETKKT
FGLESFYEGAKIEDGAETKKTFGLEGSYEGAKIEDGAETIATFGL
SKTYNGAQITD

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 13. In certain embodiments, the presently disclosed fusion protein comprises the amino acid sequence set forth in SEQ ID NO: 13. In certain embodiments, the presently disclosed fusion protein consists of the amino acid sequence set forth in SEQ ID NO: 13. SEQ ID NO: 13 is provided below.

[SEQ ID NO: 13]
AETKKTFGLEVSYDGAKIEDGAETKKTFGLEVSYDGAKIEDGGAE
TIATFGLSKTYNGAQITDGGAETKKVLGLNKNYDGAKIEDGAETK
KTFGLEVSYDGAKIEDGGAETKNSIALEKNYDGAKIEDGGAETIA
TFGLSKTYNGAQITDGAETSKKFVLENNYDGAKIEDGGAETKRTF
GLDIGYFGAKIEDGAETKKTFGLESFYEGAKIEDGAETKKTFGLE
GSYEGAKIEDGAETKKTFGLEGSYEGAKIED

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 14. In certain embodiments, the presently disclosed fusion protein comprises the amino acid sequence set forth in SEQ ID NO: 14. In certain embodiments, the presently disclosed fusion protein consists of the amino acid sequence set forth in SEQ ID NO: 14. SEQ ID NO: 14 is provided below.

[SEQ ID NO: 14]
AETKRTFGLDKNEGLTDGAETIATFGLSKTYNGAQITDGGAETIA
TFGLSKTYNGAQITDGGAETKKVLGLNKNYDGAKIEDGAETKRTF
GLDKNEGLTDGGAETIATFGLSKTYNGAQITDGGAETIATFGLSK
TYNGAQITDGAETIATFGLSKTYNGAQITDGGAETIATFGLSKTY
NGAQITDGGAETIATFGLSKTYNGAQITDGAETKKTFGLEGSYEG
AKIEDGAETIATFGLSKTYNGAQITD

In certain embodiments, the presently disclosed fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 15. In certain embodiments, the presently disclosed fusion protein comprises the amino acid sequence set forth in SEQ ID NO: 15. In certain embodiments, the presently disclosed fusion protein consists of the amino acid sequence set forth in SEQ ID NO: 15. SEQ ID NO: 15 is provided below.

[SEQ ID NO: 15]
AETKKTFGLEVSYDGAKIEDGAETIATFGLEKNYDGAKIEDGGAE
TIATFGLSKTYNGAQITDGGAETKKVLGLNKNYDGAKIEDGAETK
RTFGLDKNEGLTDGGAETKNSIALEKNYDGAKIEDGGAETIATFG
LSKTYNGAQITDGAETSKKFVLENNYDGAKIEDGGAETKRTFGLD
IGYFGAKIEDGAETKKTFGLESFYEGAKIEDGAETKKTFGLEGSY
EGAKIEDGAETIATFGLSKTYNGAQITD

In certain embodiments, the fusion protein can include the addition of an antibody epitope or other tag, to facilitate identification, targeting, and/or purification of the polypeptide. The use of 6×His (SEQ ID NO: 23) and GST (glutathione S transferase) as tags is well known. The inclusion of a cleavage site at or near the fusion junction will facilitate the removal of the extraneous polypeptide after purification. Other amino acid sequences that can be included in the polypeptide include functional domains, such as active sites from enzymes such as a hydrolase, glycosylation domains, cellular targeting signals, or transmembrane regions.

Epitope tags are well known to those of skill in the art. Moreover, antibodies specific to a wide variety of epitope tags are commercially available. These include but are not limited to antibodies against the DYKDDDDK (SEQ ID NO: 24) epitope, c-myc antibodies, the HNK-1 carbohydrate epitope, the HA epitope, the HSV epitope, the His₄ (SEQ ID NO: 24), His₅ (SEQ ID NO: 25), and His₆ (SEQ ID NO: 23) epitopes that are recognized by the His epitope-specific antibodies (see, e.g., Qiagen), and the like. In addition, vectors for epitope tagging proteins are commercially available. A polypeptide can be tagged with the FLAG® epitope (DYKDDDDK (SEQ ID NO: 22) epitope) (N-terminal, C-terminal, or internal tagging), the c-myc epitope (C-terminal), or both the FLAG (DYKDDDDK (SEQ ID NO: 22) epitope) (N-terminal) and c-myc (C-terminal) epitopes.

In certain embodiments, fusion proteins and polypeptides disclosed herein can include deletions and/or substitutions of amino acids relative to the native sequence. Sequences with amino acid substitutions are contemplated, as are sequences with a deletion, and sequences with a deletion and a substitution. In certain embodiments, these polypeptides can further include insertions or added amino acids.

Substitutional or replacement variants typically contain the exchange of one amino acid for another at one or more sites within the protein and can be designed to modulate one or more properties of the polypeptide, particularly to increase its efficacy or specificity. Substitutions of this kind can be conservative substitutions. Conservative substitution is when one amino acid is replaced with one of similar shape and charge. However, if used, conservative substitutions are well known in the art and include, for example, the changes of alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Changes other than those discussed above are generally considered not to be conservative substitutions. It is specifically contemplated that one or more of the conservative substitutions above can be included as embodiments. In certain embodiments, such substitutions are specifically excluded. Furthermore, in additional embodiments, substitutions that are not conservative are employed in variants.

In addition to a deletion or substitution, the fusion proteins can have an insertion of one or more residues. The variant amino acid sequence can be structurally equivalent to the native counterparts. For example, the variant amino acid sequence forms the appropriate structure and conformation for binding targets, proteins, or polypeptide segments.

The following is a discussion based on changing of the amino acids of a polypeptide (e.g., a presently disclosed fusion protein) to create a mutant molecule. For example, certain amino acids can be substituted for other amino acids in a polypeptide without appreciable loss of function, such as the ability to interact with an antibody or a target polypeptide sequence. Since it is the interactive capacity and nature of a polypeptide that defines that polypeptide's functional activity, certain amino acid substitutions can be made in a polypeptide sequence and nevertheless produce a polypeptide with like properties. In making such changes, the hydropathic index of amino acids can be considered. The importance of the hydropathic amino acid index in conferring interactive function on a protein is generally understood in the art. See Kyte and Doolittle (1982) J. Mol. Biol. 157:105-132. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of amino acids can be made effectively based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5=1); alanine (0.5); histidine (−0.5); cysteine (−1.0); methionine (−1.3); valine (−1.5); leucine (−1.8); isoleucine (−1.8); tyrosine (2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within +2, within +1, and within +0.5. As outlined above, amino acid substitutions generally are based on the relative similarity of the amino acid side-chain substituents, for example, their hydrophobicity, hydrophilicity, charge, size, and the like. However, in certain embodiments, a non-conservative substitution is contemplated. In certain embodiments, a random substitution is also contemplated. Exemplary substitutions that take into consideration the various foregoing characteristics are well known to those of skill in the art and include arginine and lysine; glutamate and aspartate; serine and threonine; glutamine and asparagine; and valine, leucine, and isoleucine.

Techniques for determining nucleic acid and amino acid sequence identity are known in the art. Typically, such techniques include determining the nucleotide sequence of the mRNA for a gene and/or determining the amino acid sequence encoded thereby and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. Unless indicated otherwise, percent identity is determined for two sequences when compared and aligned for maximum correspondence over a comparison window or designated region as measured using a BLAST or BLAST 2.0 sequence comparison algorithms with default parameters. See, e.g., the NCBI website at ncbi.nlm.nih.gov/BLAST. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

### 2.2. Host Cells

The present disclosure also provides recombinant microorganisms comprising the presently disclosed fusion proteins (e.g., fusion proteins set forth in SEQ ID Nos: 10-17).

In certain embodiments, the microorganism is a bacterium. In certain embodiments, the microorganism is selected from the group consisting of Aceiobacter aceti, Achromobacter, Acidiphilium, Acinetobacter, Actinomadura, Actinoplanes, Aeropyrumpernix, Agrobacterium. Alcaligenes, Ananas comosus (M), Arthrobacter, Bacillus alcalophilus, Bacillus amyloliquefaciens, Bacillus brevis, Bacillus circulars, Bacillus clausii, Bacillus lentus, Bacillus lichenifirmis, Bacillus macerans, Bacillus stearothermophilus, Bacillus subtilis, Bifidobacterium, Brevibacillus brevis, Burkholderia cepacia, Candida cylindracea, Carica papaya (L), Cellulosimicrobium. Cephalosporium, Chaetomium erraticum, Chaetomium gracile. Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter. Gluconacetobacter, Haloarcula. Humicola insolens, Kitasatospora setae. Klebsiella, Klebsiella oxytoca, Kocuria, Lactlactis, Lactobacillus. Lactobacillus fermentum, Lactobacillus sake, Lactococcus, Lactococcus lactis. Leuconostoc, Methylocystis, Methanolobus siciliae. Methanogenium organophilum. Methanobacterium bryantii, Microbacterium imperiale, Micrococcus lysodeikticus, Microlunatus, Mucorjavanicus, Mycobacterium, Myrothecium, Nitrobacter, Nitrosomonas, Nocardia, Papaya carica, Pediococcus. Pediococcus halophilus, Paracoccus pantotrophus, Propionibacterium, Pseudomonas, Pseudomonasfluorescens, Pseudomonas denitrificans, Pyrococcus, Pyrococcusfuriosus, Pyrococcus horikoshii, Rhizobium, Rhizomucor miehei, Rhizomucor pusillus Lindt, Rhizopus, Rhizopus delemar, Rhizopus japonicas, Rhizopus niveus. Rhizopus oryzae, Rhizopus oligosporus. Rhodococcus, Sckroiina libertina, Sphingobacterium multivorum, Sphingobium, Sphingomonas, Streptococcus. Streptococcus thermophilus Y-1, Streptomyces, Streptomyces griseus, Streptomyces lividans, Streptomyces murinus, Streptomyces ruhiginosus. Streptomyces violaceoruber, Streptoverticillium mobaraense, Tetragenococcus. Thermus. Thiosphaera pantotropha, Trametes, Vibrio alginolyticus, Xanthomonas, Zymomonas, and Zymomonus mobilis. In certain embodiments, the microorganism is Escherichia coli (E. coli).

In certain embodiments, the E. coli is selected from the group consisting of Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli 0157:H7, E. coli O104:H4, E. coli O121, E. coli O104:H21, E. coli Kl, and E. coli NC101.

In certain embodiments, the E. coli is derived from a strain selected from the group consisting of NCTC 12757, NCTC 12779, NCTC 12790, NCTC 12796, NCTC 12811, ATCC 11229, ATCC 25922, ATCC 8739, DSM 30083, BC 5849, BC 8265, BC 8267, BC 8268, BC 8270, BC 8271, BC 8272, BC 8273, BC 8276, BC 8277, BC 8278, BC 8279, BC 8312, BC 8317, BC 8319, BC 8320, BC 8321, BC 8322, BC 8326, BC 8327, BC 8331, BC 8335, BC 8338, BC 8341, BC 8344, BC 8345, BC 8346, BC 8347, BC 8348, BC 8863, and BC 8864.

In certain embodiments, the E. coli is derived from a strain selected from the group consisting of BC 4734 (O26:H11), BC 4735 (O157:H-), BC 4736, BC 4737 (n.d.), BC 4738 (O157:H7), BC 4945 (O26:H-), BC 4946 (O157:H7), BC 4947 (O111:H-), BC 4948 (O157:H), BC 4949 (O5), BC 5579 (O157:H7), BC 5580 (O157:H7), BC 5582 (O3:H), BC 5643 (O2:H5), BC 5644 (O128), BC 5645 (O55:H-), BC 5646 (O69:H-), BC 5647 (O101:H9), BC 5648 (O103:H2), BC 5850 (O22:H8), BC 5851 (O55:H-), BC 5852 (O48:H21), BC 5853 (O26:H11), BC 5854 (O157:H7), BC 5855 (O157:H-), BC 5856 (O26:H-), BC 5857 (O103:H2), BC 5858 (O26:H11), BC 7832, BC 7833 (O raw form:H-), BC 7834 (ONT:H-), BC 7835 (O103:H2), BC 7836 (O57:H-), BC 7837 (ONT:H-), BC 7838, BC 7839 (O128:H2), BC 7840 (O157:H-), BC 7841 (O23:H-), BC 7842 (O157:H-), BC 7843, BC 7844 (O157:H-), BC 7845 (O103:H2), BC 7846 (O26:H11), BC 7847 (O145:H-), BC 7848 (O157:H-), BC 7849 (O156:H47), BC 7850, BC 7851 (O157:H-), BC 7852 (O157:H-), BC 7853 (O5:H-), BC 7854 (O157:H7), BC 7855 (O157:H7), BC 7856 (O26:H-), BC 7857, BC 7858, BC 7859 (ONT:H-), BC 7860 (O129:H-), BC 7861, BC 7862 (O103:H2), BC 7863, BC 7864 (O raw form:H-), BC 7865, BC 7866 (O26:H-), BC 7867 (O raw form:H-), BC 7868, BC 7869 (ONT:H-), BC 7870 (O113:H-), BC 7871 (ONT:H-), BC 7872 (ONT:H-), BC 7873, BC 7874 (O raw form:H-), BC 7875 (O157:H-), BC 7876 (O111:H-), BC 7877 (O146:H21), BC 7878 (O145:H-), BC 7879 (O22:H8), BC 7880 (O raw form:H-), BC 7881 (O145:H-), BC 8275 (O157:H7), BC 8318 (O55:K-:H-), BC 8325 (O157:H7), BC 8332 (ONT), and BC 8333.

In certain embodiments, the E. coli is derived from a strain selected from the group consisting of BC 8246 (O152:K-:H-), BC 8247 (O124:K(72):H3), BC 8248 (O124), BC 8249 (O112), BC 8250 (O136:K(78):H-), BC 8251 (O124:H-), BC 8252 (O144:K-:H-), BC 8253 (O143:K:H-), BC 8254 (O143), BC 8255 (O112), BC 8256 (O28a.e), BC 8257 (O124:H-), BC 8258 (O143), BC 8259 (O167:K-:H5), BC 8260 (O128a.c.:H35), BC 8261 (O164), BC 8262 (O164:K-:H-), BC 8263 (O164), and BC 8264 (O124).

In certain embodiments, the E. coli is derived from a strain selected from the group consisting of BC 5581 (O78:H11), BC 5583 (O2:K1), BC 8221 (O118), BC 8222 (O148:H-), BC 8223 (O111), BC 8224 (O110:H-), BC 8225 (O148), BC 8226 (O118), BC 8227 (O25:H42), BC 8229 (O6), BC 8231 (O153:H45), BC 8232 (O9), BC 8233 (O148), BC 8234 (O128), BC 8235 (O118), BC 8237 (O111), BC 8238 (O110:H17), BC 8240 (O148), BC 8241 (O6H16), BC 8243 (O153), BC 8244 (O15:H-), BC 8245 (O20), BC 8269 (O125a.c:H-), BC 8313 (O6:H6), BC 8315 (O153:H-), BC 8329, BC 8334 (O118:H12), and BC 8339.

In certain embodiments, the E. coli is derived from a strain selected from the group consisting of BC 7567 (O86), BC 7568 (O128), BC 7571 (O114), BC 7572 (O119), BC 7573 (O125), BC 7574 (O124), BC 7576 (O127a), BC 7577 (O126), BC 7578 (O142), BC 7579 (O26), BC 7580 (OK26), BC 7581 (O142), BC 7582 (O55), BC 7583 (O158), BC 7584 (O-), BC 7585 (O-), BC 7586 (O-), BC 8330, BC 8550 (O26), BC 8551 (O55), BC 8552 (O158), BC 8553 (O26), BC 8554 (O158), BC 8555 (O86), BC 8556 (O128), BC 8557 (OK26), BC 8558 (O55), BC 8560 (O158), BC 8561 (O158), BC 8562 (O114), BC 8563 (O86), BC 8564 (O128), BC 8565 (O158), BC 8566 (O158), BC 8567 (O158), BC 8568 (O111), BC 8569 (O128), BC 8570 (O114), BC 8571 (O128), BC 8572 (O128), BC 8573 (O158), BC 8574 (O158), BC 8575 (O158), BC 8576 (O158), BC 8577 (O158), BC 8578 (O158), BC 8581 (O158), BC 8583 (O128), BC 8584 (O158), BC 8585 (O128), BC 8586 (O158), BC 8588 (O26), BC 8589 (O86), BC 8590 (O127), BC 8591 (O128), BC 8592 (O114), BC 8593 (O114), BC 8594 (O114), BC 8595 (O125), BC 8596 (O158), BC 8597 (O26), BC 8598 (O26), BC 8599 (O158), BC 8605 (O158), BC 8606 (O158), BC 8607 (O158), BC 8608 (O128), BC 8609 (O55), BC 8610 (O114), BC 8615 (O158), BC 8616 (O128), BC 8617 (O26), BC 8618 (O86), BC 8619, BC 8620, BC 8621, BC 8622, BC 8623, BC 8624 (O158), and BC 8625 (O158).

In certain embodiments, the microorganism is a fungal cell. In certain embodiments, the fungal cell is selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromyces fragilis. Kluyveromyces lactis. Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillaium, Yarrowia lipolytica, and Zygosaccharomyces rouxii.

In certain embodiments, the microorganism is a yeast cell. In certain embodiments, the yeast cell is Saccharomyces cerevisiae.

### 2.2. Methods of Preparation

The present disclosure also provides methods for producing the presently disclosed fusion proteins. In certain embodiments, the presently disclosed methods for producing the fusion protein include culturing microorganisms (e.g., one disclosed in above) and purifying the fusion protein.

As used herein, “culturing” a cell refers to introducing an appropriate culture medium, under appropriate conditions, to promote the growth of a cell. In certain embodiments, culturing is performed using a liquid or solid growth medium. In certain embodiments, culturing occurs under aerobic or anaerobic conditions based on the requirements of the microorganism and the desired metabolic state of the same. In certain embodiments, culturing includes specific conditions such as temperature, pressure, light, pH, and cell density.

In certain embodiments, the methods for producing the fusion protein include a culture medium for culturing the recombinant bacteria. “Culture medium,” as used herein, refers to any composition or broth that supports the growth of the microorganism disclosed herein. A culture media can be liquid or solid. In certain embodiments, the culture media includes nutrients, salts, buffers, elements, and other compounds that support the growth and viability of cells. Additionally, culture media can include sources of nitrogen, carbon, amino acids, carbohydrates, trace elements, vitamins, and minerals. In certain embodiments, the culture media includes a complex extract (e.g., yeast extract). In certain embodiments, the culture medium is enriched in order to support rapid growth. In certain embodiments, the culture medium is modified in order to support slower growth. In certain embodiments, the culture medium includes an agent that can inhibit the growth of or kill contaminating organisms (e.g., an antibiotic). In certain embodiments, the culture medium includes an agent that can activate an inducible promoter or enzyme (e.g., IPTG). Non-limiting examples of culture media encompassed by the present disclosure include M9 medium, Lysogeny Broth (LB), Terrific Broth (TB), and YT broth.

The fusion proteins disclosed herein can be purified by any suitable method. Such methods include but are not limited to, the use of affinity matrices or hydrophobic interaction chromatography. For example, without any limitation, a Protein A, Protein G, Protein A/G, or an antibody affinity column can be used to bind and/or to purify the fusion protein. Hydrophobic interactive chromatography, for example, a butyl or phenyl column, can also be suitable for purifying some polypeptides. Ion exchange chromatography (for example anion exchange chromatography and/or cation exchange chromatography) can also be suitable for purifying some polypeptides. Mixed-mode chromatography (for example reversed phase/anion exchange, reversed phase/cation exchange, hydrophilic interaction/anion exchange, hydrophilic interaction/cation exchange, etc.) can also be suitable for purifying some polypeptides. Many methods of purifying polypeptides are known in the art.

In certain embodiments, the fusion protein is produced in a cell-free system. Nonlimiting exemplary cell-free systems are described, for example, in Sitaraman et al., Methods Mol. Biol. 498:229-44 (2009); Spirin, Trends Biotechnol. 22:538-45 (2004); Endo et al., Biotechnol. Adv. 21:695-713 (2003).

### 2.3. Nucleic Acid Molecules and Vectors Encoding Fusion Proteins

Nucleic acid molecules comprising polynucleotides that encode the presently disclosed fusion proteins are provided herein. In certain embodiments, the polynucleotide comprises a nucleotide sequence that encodes a leader sequence. In certain embodiments, the leader sequence is a native leader sequence. In certain embodiments, the leader sequence is a heterologous leader sequence.

In certain embodiments, the presently disclosed nucleic acid molecule comprises a nucleotide sequence that is at least about 80%, about 85%, about 90%, about 91%, about 92%, about 93%, about 94%, or about 95%, about 96%, about 97%, about 98%, or about 99% identical to the nucleotide sequence set forth in SEQ ID NO: 18. In certain embodiments, the presently disclosed nucleic acid molecule comprises the nucleotide sequence set forth in SEQ ID NO: 18. In certain embodiments, the presently disclosed nucleic acid molecule consists of the nucleotide sequence set forth in SEQ ID NO: 18. SEQ ID NO: 18 is provided below.

[SEQ ID NO: 18]
GCGGAAACCAAGAAAACCTTTGGTCTGGAAGTGAGCTATGACGGC
GCGAAAATCGAAGATGGTGGCGCGGAGACCATTGCGACCTTCGGT
CTGAGCAAGACCTACAACGGTGCGCAGATCACCGATGGTGGCGCG
GAAACCAAGAAAGTTCTGGGTCTGAACAAAAACTATGACGGTGCG
AAGATTGAAGATGGCGCGGAGACCAAACGTACCTTTGGTCTGGAT
AAGAACGAAGGTCTGACCGATGGTGGCGCGGAGACTAAAAACAGC
ATCGCGCTGGAGAAGAACTACGATGGTGCGAAGATCGAGGACGGT
GGCGCGGAAACCATCGCGACCTTCGGCCTGAGCAAAACCTATAAC
GGTGCGCAAATCACCGACGGCGCGGAGACCAGCAAGAAATTTGTG
CTGGAAAACAACTATGACGGTGCGAAAATTGAGGACGGTGGCGCG
GAGACCAAGCGTACCTTCGGCCTGGATATCGGTTACTTTGGCGCG
AAAATTGAAGACGGTGCGGAGACCAAGAAAACCTTCGGCCTGGAA
AGCTTTTATGAGGGTGCGAAGATCGAGGATGGTGCTGAAACCAAG
AAAACCTTCGGTCTGGAAGGCAGCTACGAGGGTGCGAAAATCGAA
GACGGCGCGGAAACCATTGCGACCTTTGGTCTGAGCAAGACCTAT
AACGGCGCGCAGATTACCGATTAA

In certain embodiments, the presently disclosed nucleic acid molecule comprises a nucleotide sequence that is at least about 80%, about 85%, about 90%, about 91%, about 92%, about 93%, about 94%, or about 95%, about 96%, about 97%, about 98%, or about 99% identical to the nucleotide sequence set forth in SEQ ID NO: 19. In certain embodiments, the presently disclosed nucleic acid molecule comprises the nucleotide sequence set forth in SEQ ID NO: 19. In certain embodiments, the presently disclosed nucleic acid molecule consists of the nucleotide sequence set forth in SEQ ID NO: 19. SEQ ID NO: 19 is provided below.

[SEQ ID NO: 19]
AGCAACATTGGTGCGGAAACCAAGAAAACCTTTGGTCTGGAAGTG
AGCTATGACGGCGCGAAAATCGAAGATGGTGGCGCGGAGACCATT
GCGACCTTCGGTCTGAGCAAGACCTACAACGGTGCGCAGATCACC
GATGGTGGCGCGGAAACCAAGAAAGTTCTGGGTCTGAACAAAAAC
TATGACGGTGCGAAGATTGAAGATGGCGCGGAGACCAAACGTACC
TTTGGTCTGGATAAGAACGAAGGTCTGACCGATGGTGGCGCGGAG
ACTAAAAACAGCATCGCGCTGGAGAAGAACTACGATGGTGCGAAG
ATCGAGGACGGTGGCGCGGAAACCATCGCGACCTTCGGCCTGAGC
AAAACCTATAACGGTGCGCAAATCACCGACGGCGCGGAGACCAGC
AAGAAATTTGTGCTGGAAAACAACTATGACGGTGCGAAAATTGAG
GACGGTGGCGCGGAGACCAAGCGTACCTTCGGCCTGGATATCGGT
TACTTTGGCGCGAAAATTGAAGACGGTGCGGAGACCAAGAAAACC
TTCGGCCTGGAAAGCTTTTATGAGGGTGCGAAGATCGAGGATGGT
GCTGAAACCAAGAAAACCTTCGGTCTGGAAGGCAGCTACGAGGGT
GCGAAAATCGAAGACGGCGCGGAAACCATTGCGACCTTTGGTCTG
AGCAAGACCTATAACGGCGCGCAGATTACCGATTAA

In certain embodiments, the presently disclosed nucleic acid molecule comprises a nucleotide sequence that is at least about 80%, about 85%, about 90%, about 91%, about 92%, about 93%, about 94%, or about 95%, about 96%, about 97%, about 98%, or about 99% identical to the nucleotide sequence set forth in SEQ ID NO: 20. In certain embodiments, the presently disclosed nucleic acid molecule comprises the nucleotide sequence set forth in SEQ ID NO: 20. In certain embodiments, the presently disclosed nucleic acid molecule consists of the nucleotide sequence set forth in SEQ ID NO: 20. SEQ ID NO: 20 is provided below.

[SEQ ID NO: 20]
GCGGAGACCAAGAAAACCTTTGGCCTGGAAGTGAGCTATGACGGT
GCGAAGATTGAGGATGGCGCGGAAACCAAGAAAACCTTCGGTCTG
GAAGTGAGCTACGACGGCGCGAAGATCGAGGATGGTGGCGCGGAA
ACCATTGCGACCTTTGGTCTGAGCAAAACCTATAACGGCGCGCAA
ATTACCGACGGTGGCGCGGAGACCAAGAAAGTGCTGGGTCTGAAC
AAGAACTACGACGGTGCGAAAATCGAAGACGGTGCTGAGACCAAG
AAAACCTTCGGCCTGGAAGTGAGCTATGACGGGGCGAAAATTGAA
GACGGTGGCGCGGAAACCAAAAACAGCATTGCGCTGGAAAAGAAC
TATGATGGTGCGAAAATTGAAGATGGTGGCGCGGAGACCATTGCG
ACCTTCGGCCTGAGCAAAACCTACAACGGTGCGCAAATCACCGAT
GGTGCGGAGACCAGCAAGAAATTTGTGCTGGAAAACAATTACGAT
GGCGCGAAGATTGAGGACGGTGGCGCGGAGACTAAACGTACCTTC
GGTCTGGACATCGGTTACTTCGGTGCGAAAATTGAGGATGGTGCT
GAAACCAAGAAAACCTTTGGTCTGGAGAGCTTTTATGAAGGTGCG
AAAATCGAGGACGGTGCCGAGACCAAGAAAACCTTCGGGCTGGAG
GGCAGCTACGAAGGCGCGAAAATCGAAGACGGAGCTGAGACCAAG
AAAACCTTCGGACTGGAGGGCAGCTATGAAGGCGCGAAGATCGAA
GACTAA

In certain embodiments, the presently disclosed nucleic acid molecule comprises a nucleotide sequence that is at least about 80%, about 85%, about 90%, about 91%, about 92%, about 93%, about 94%, or about 95%, about 96%, about 97%, about 98%, or about 99% identical to the nucleotide sequence set forth in SEQ ID NO: 21. In certain embodiments, the presently disclosed nucleic acid molecule comprises the nucleotide sequence set forth in SEQ ID NO: 21. In certain embodiments, the presently disclosed nucleic acid molecule consists of the nucleotide sequence set forth in SEQ ID NO: 21. SEQ ID NO: 21 is provided below.

[SEQ ID NO: 21]
GCGGAAACCAAGCGTACCTTTGGCCTGGACAAAAACGAGGGTCTG
ACCGATGGCGCGGAAACCATTGCGACCTTTGGTCTGAGCAAGACC
TATAACGGTGCGCAGATCACCGATGGTGGCGCGGAGACCATTGCG
ACCTTCGGTCTGAGCAAAACCTACAACGGTGCGCAAATTACCGAT
GGTGGCGCGGAAACCAAGAAAGTGCTGGGTCTGAACAAGAACTAT
GACGGTGCGAAAATTGAGGATGGCGCGGAGACCAAACGTACCTTC
GGTCTGGACAAAAACGAGGGCCTGACCGACGGTGGCGCGGAGACC
ATCGCGACCTTCGGCCTGAGCAAGACCTACAACGGCGCGCAAATC
ACCGACGGTGGCGCGGAAACCATCGCGACCTTTGGCCTGAGCAAA
ACCTATAATGGCGCGCAGATCACCGACGGTGCTGAGACTATCGCT
ACTTTTGGCCTGAGCAAGACCTATAATGGCGCGCAAATAACTGAT
GGTGGCGCGGAGACTATCGCTACTTTCGGTCTGAGCAAGACCTAC
AATGGTGCGCAAATCACTGATGGTGGCGCGGAAACTATCGCTACT
TTTGGTCTGAGCAAAACCTATAACGGTGCGCAAATCACCGACGGC
GCGGAGACCAAGAAAACCTTTGGTCTGGAGGGCAGCTATGAAGGT
GCGAAGATCGAGGACGGTGCTGAAACCATCGCTACTTTCGGATTA
AGCAAAACCTATAACGGCGCGCAGATTACCGACTAA

Nucleic acid molecules can be constructed using recombinant DNA techniques conventional in the art. In certain embodiments, a nucleic acid molecule is an expression vector that is suitable for expression in a selected host cell (e.g., a host cell disclosed in Section 2.2).

Vectors comprising polynucleotides that encode the presently disclosed fusion proteins are also provided. Such vectors include, but are not limited to, DNA vectors, phage vectors, viral vectors, retroviral vectors, etc. In certain embodiments, a vector is selected that is designed for the expression of polypeptides in bacterial cells. For example, but without any limitation, a vector that is designed for the expression of polypeptides in E. coli cells is selected.

Introduction of one or more nucleic acids into a desired host cell can be accomplished by any method including, without any limitation, calcium phosphate transfection, DEAE-dextran mediated transfection, cationic lipid-mediated transfection, electroporation, transduction, infection, etc. Additional non-limiting methods are described in Sambrook et al., Molecular Cloning, A Laboratory Manual, 3^{rd} ed. Cold Spring Harbor Laboratory Press (2001). In certain embodiments, the nucleic acids can be transiently transfected in the desired host cells. In certain embodiments, the nucleic acids can be stably transfected in the desired host cells.

### 2.4. Compositions

In certain embodiments, the present disclosure provides fusion proteins prepared by the methods described above. In certain embodiments, the fusion protein is prepared in a host cell. In certain embodiments, the fusion protein is prepared in a cell-free system. In certain embodiments, the fusion protein is purified. In certain embodiments, a cell culture media comprising the fusion protein is provided.

In certain embodiments, compositions comprising the fusion proteins disclosed herein are provided. In certain embodiments, the composition comprises the fusion protein. In certain embodiments, the composition comprises the host cell comprising the fusion protein. In certain embodiments, the composition comprises the nucleic acid encoding the fusion protein. In certain embodiments, the composition comprises the vector encoding the fusion protein.

In certain embodiments, compositions disclosed herein further comprise a second fusion protein including a combination of polypeptides that can be recognized by antibodies targeting Ehrlichia canis.

In certain embodiments, the second fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 16. In certain embodiments, the presently disclosed fusion protein comprises the amino acid sequence set forth in SEQ ID NO: 16. In certain embodiments, the presently disclosed fusion protein consists of the amino acid sequence set forth in SEQ ID NO: 16. SEQ ID NO: 16 is provided below.

[SEQ ID NO: 16]
SNIGTEDSVSAPATEDSVSAPATEDSVSAPATEDSVSAPATEDSV
SAPATEDSVSAPAGGGSYNHNTGLLDLDSDILNMLYSYNHNTGLL
DLDSDILNMLYSYNHNTGLLDLDSDILNMLYGGGSKEESTPEVKA
EDLQPAVDSKEESTPEVKAEDLQPAVDSKEESTPEVKAEDLQPAV
D

In certain embodiments, compositions disclosed herein further comprise a third fusion protein including a combination of polypeptides that can be recognized by antibodies targeting Ehrlichiae spp.

In certain embodiments, the third fusion protein comprises an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 17. In certain embodiments, the presently disclosed fusion protein comprises the amino acid sequence set forth in SEQ ID NO: 17. In certain embodiments, the presently disclosed fusion protein consists of the amino acid sequence set forth in SEQ ID NO: 17. SEQ ID NO: 17 is provided below.

[SEQ ID NO: 17]
SNIGNTTTGVFGLKQDWDGATIKDGGNTTVGVFGLKQNWDGSAIS
NGGNPTVALYGLKQDWNGVSAGGNTTVGVFGIEQDWDRCVISGGN
PTVALYGLKQDWEGISSGGKSTVGVFGLKHDWDGSPILKGGNTTT
GVFGLKQDWDGSTISGGNTTTGVFGLKQDWDGATIKDGGNPTVAL
YGLKQDWNGVSAGGKSTVGVFGLKHDWDGSPILKGGNTTVGVFGI
EQDWDRCVISGGNTTTGVFGLKQDWDGATIKDGGNTTVGVFGLKQ
NWDGSAISNGGKSTVGVFGLKHDWDGSPILK

### 3. Methods of Detection

The present disclosure further provides methods for detecting various infectious organisms including E. ewingii in a subject. In certain embodiments, the method can be used for diagnosis, prognosis, stratification, risk assessment, or treatment monitoring of a disease.

In certain embodiments, the present disclosure provides methods for detecting an antibody that specifically binds to an E. ewingii polypeptide in a sample. In certain embodiments, the method comprises contacting the fusion protein disclosed herein with said sample and detecting a complex formed between the antibody and the fusion protein. In certain embodiments, the method comprises contacting the compositions disclosed herein (e.g., described in Section 2.4) with said sample and detecting a complex formed between the antibody and the composition.

In certain embodiments, the present disclosure provides methods of identifying a subject having an E. ewingii infection. In certain embodiments, the method comprises contacting the fusion protein disclosed herein with a sample from the subject. Additionally or alternatively, the method comprises contacting the compositions or solid supports disclosed herein (e.g., described in Section 2.4) with said sample. In certain embodiments, the method comprises detecting a complex formed between the sample and the fusion protein, the composition, or the solid support. In certain embodiments, an increased level of detected complexes relative to a normal control indicates that the subject has an E. ewingii infection.

In certain embodiments, the method can be used to detect Lyme disease. In certain embodiments, the method can be used for the classification of Lyme exposure of a subject, e.g., an animal by calculating levels of antibodies that specifically bind to the fusion protein disclosed herein using a method for detecting an antibody that specifically binds to an E. ewingii antigen in a sample disclosed herein; calculating reference values of the levels of the antibodies, and determining the type of E. ewingii infection of the subject by comparing the levels of the antibodies to the reference values. The reference values can be calculated using levels of detectable signals of negative controls, and more than one reference value may be calculated for each antibody that specifically binds to a Lyme polypeptide.

The reference values can be established by analyzing results from experimental samples from animals that are infected with or vaccinated against E. ewingii. Empirical values can be calculated from the analysis of experimental samples and used for the calculation of reference values for the antibodies. Initially, artificial values can be set for each reference value and adjusted by an algorithm using experimental data. A minimal value for each reference value can also be established from the analysis of experimental samples and in cases where the reference value calculated is less than the minimal value, the minimal value can be used.

Further provided herein is a method for detecting multiple disease antigens and/or antibodies in a sample, which method comprises a) contacting said sample with a composition for detecting multiple disease antigens and/or antibodies comprising the presently disclosed fusion proteins; and b) detecting a complex formed between the antibody and the fusion protein.

In certain embodiments, the sample may be from a subject selected from the group consisting of a dog, a cat, a human, and a horse. In certain embodiments, the method can be used for diagnosis, prognosis, stratification, risk assessment, or treatment monitoring of a disease. In certain embodiments, the sample can be selected from the group consisting of a serum, a plasma, and a blood sample. In certain embodiments, the sample may be a clinical sample. In some embodiments, the antibody can be a monoclonal or polyclonal antibody or antibody fragment.

The detection of antibodies and/or antigens can be achieved by immunoassays, including any immunoassay known in the art including, but not limited to, radioimmunoassay, enzyme-linked immunosorbent assay (ELISA), “sandwich” assay, precipitin reaction, agglutination assay, fluorescent immunoassay, and chemiluminescence-based immunoassay. In certain embodiments, the complex formed between the antibody and the fusion protein can be assessed by a sandwich or competitive assay format, optionally with a binder or antibody. In certain embodiments, the binder or antibody can be attached to a surface and functions as a capture antibody. In certain embodiments, the capture binder or antibody can be attached to the surface directly or indirectly. In some embodiments, the binder or antibody can be attached to the surface via a biotin-avidin (or streptavidin) linking pair. In certain embodiments, at least one of the binders or antibodies can be labeled. In certain embodiments, the complex formed between the antibody and the fusion protein can be assessed by a format selected from the group consisting of an enzyme-linked immunosorbent assay (ELISA), Western blotting, immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay and avidity assay. In certain embodiments, the complex formed between the antibody and the fusion protein can be assessed in a homogeneous or heterogeneous assay format.

In certain embodiments, multiple reagents for detecting infectious organisms can be included in the same assay, such as parallel immunoassay. For example, but without any limitation, a parallel immunoassay can include at least about 2, about 3, about 4, about 5, about 10, about 100, about 1000 or more reagents, such as antibodies or antigenic polypeptides, in the same assay system.

Numerous technological platforms for performing parallel immunoassays are known. Generally, such methods involve a logical or physical array of either the subject samples, the protein markers, or both. Common array formats include both liquid and solid phase arrays. For example, assays employing liquid phase arrays, e.g., for hybridization of nucleic acids, binding of antibodies or other receptors to ligands, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., the ORCA™ system from Beckman-Coulter, Inc. (Fullerton, Calif.) and the Zymate systems from Zymark Corporation (Hopkinton, Mass.).

Additionally or alternatively, a variety of solid phase arrays can favorably be employed for parallel immunoassays in the context of the invention. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly) vinylidenedifluoride, polystyrene, polycarbonate, or combinations thereof can all serve as the substrate for a solid phase array.

The polypeptides/antibodies can be immobilized to solid phase support for the detection of antibody binding. As used herein, “solid phase support” is not limited to a specific type of support. Rather a large number of supports are available and are known to one of ordinary skill in the art. Solid phase supports include silica gels, resins, derivatized plastic films, glass beads, cotton, plastic beads, and alumina gels. A suitable solid phase support may be selected based on desired end use and suitability for various synthetic protocols. For example, for polypeptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TENTAGEL®, Rapp Polymere, Tubingen, Germany) or polydimethylacrylamide resin (obtained from Milligen/Biosearch, California). In certain embodiments, solid phase support refers to polydimethylacrylamide resin.

In certain embodiments, the array can be a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof may be affixed to the chip in a logically ordered manner, i.e., in an array. Detailed discussions of methods for linking nucleic acids and proteins to a chip substrate are found in, e.g., U.S. Pat. Nos. 5,143,854, 5,837,832, 6,087,112, 5,215,882, 5,707,807, 5,807,522, 5,958,342, 5,994,076, 6,004,755, 6,048,695, 6,060,240, 6,090,556, and 6,040,138, each of which is hereby incorporated in its entirety.

Microarray signals can be detected by scanning the microarray with a variety of laser or CCD-based scanners and extracting features with numerous software packages, for example, Imagene (Biodiscovery), Feature Extraction Software (Agilent), Scanalyze (Eisen, M. 1999. SCANALYZE User Manual; Stanford Univ., Stanford, Calif. Ver 2.32.), GenePix (Axon Instruments).

High-throughput protein systems include commercially available systems from Ciphergen Biosystems, Inc. (Fremont, Calif.) such as PROTEIN CHIP® arrays and the Schleicher and Schuell protein microspot array (FastQuant Human Chemokine, S&S Bioscences Inc., Keene, N.H., US). In one embodiment, the high-throughput protein assay system may be the Bio-CD system using the SDI™ (Spinning Disc Interferometry) technology by Quadraspec, Inc. (West Lafayette, Ind.). Detailed discussions of the Bio-CD system are found in, e.g., U.S. Pat. Nos. 6,685,885, 7,405,831, 7,552,282, 7,659,968, 7,663,092, 7,787,126, 7,910,356, U.S. Pat. Pub. No. 2004/0166593, U.S. Pat. Pub. No. 2006/0256676, U.S. Pat. Pub. No. 2007/0023643, U.S. Pat. Pub. No. 2007/0212257, U.S. Pat. Pub. No. 2007/0259366, U.S. Pat. Pub. No. 2008/0175755, U.S. Pat. Pub. No. 2009/0002716, U.S. Pat. Pub. No. 2009/0263913, U.S. Pat. Pub. No. 2010/0145627, and Canadian Pat. Pub. No. 2681722, each of which is hereby incorporated in its entirety.

The parallel immunoassay results obtained as described above can then be used for diagnosis of the specific disorder. The individual proteins/antibodies can be detected or quantified by any of several means well known to those of skill in the art. In certain embodiments, a qualitative change in one or more proteins/antibodies is determined. Qualitative changes include the appearance of a proteins/antibodies spot that is not detectable in samples obtained from normal controls or the disappearance of a proteins/antibodies spot that is detectable in normal controls but not in the sample taken from an affected subject.

In certain embodiments, a quantitative change in one or more proteins/antibodies may be measured. The concentration of protein/antibody levels may be expressed in absolute terms, for example, optical density as read by image analysis. Additionally or alternatively, the concentrations can be expressed as a fraction, relative to normal levels of the same protein/antibody.

### 4. Computer Readable Medium

In certain non-limiting embodiments, the present disclosure provides a computer-readable medium containing executable instructions that when executed perform a method of classifying E. ewingii infection of a subject, e.g., an animal, the method comprising: calculating levels of antibodies that specifically bind to the presently disclosed fusion protein using a method for detecting an antibody that specifically binds to an E. ewingii antigen in a sample; calculating reference values of the levels of the antibodies; and determining the type of E. ewingii infection of the subject by comparing the levels of the antibodies to the reference values.

Further provided herein is a system for classifying E. ewingii infection of a subject, e.g., an animal comprising the computer-readable medium disclosed herein and an antigenic composition comprising the fusion protein disclosed herein.

### 5. Kits

In certain embodiments, the present disclosure provides kits for detecting infectious organisms. In certain embodiments, the kit includes, in a container, the polypeptides or antigenic compositions disclosed herein. In certain embodiments, the container is a sealed container. Non-limiting examples of containers include a microtiter plate, a bottle, a metal tube, a laminate tube, a plastic tube, a dispenser, a pressurized container, a barrier container, a package, a compartment, or other types of containers such as injection or blow-molded plastic containers into which the dispersions or compositions or desired bottles, dispensers, or packages are retained. Further examples of containers include glass or plastic vials or bottles.

In certain embodiments, the containers can dispense or contain a pre-determined amount of a composition of the present invention. For example, but without any limitation, the composition can be dispensed as a liquid, a fluid, or a semi-solid. In certain embodiments, the kit can also include instructions for using the kit and/or compositions. In certain embodiments, the instructions can include an explanation of how to use and maintain the compositions.

### 6. Methods of Treatment

The present disclosure provides for methods of preventing, treating, and/or delaying progression of E. ewingii infection of a subject in need thereof. In certain embodiments, the subject is a dog. In certain embodiments, the method comprises administering an antibiotic to the subject. Non-limiting examples of antibiotics to be administered to the dog include clindamycin, metronidazole, amoxicillin, cephalexin, doxycycline, enrofloxacin, gentamicin, trimethoprim, sulfamethoxazole, clavamox, chloramphenicol, cefpodoxime, tetracycline, marbofloxacin, antirobe, ciprofloxacin, albon, amoxicillin, baytril, biomox amoxicillin, cephalexin, amoxicillin, clavulanic acid, ketoconazole, and sulfadimethoxine. In certain embodiments, the antibiotic is doxycycline.

In certain embodiments, the antibiotic is administered at an amount of about 1 mg/kg to about 100 mg/kg. For example, and not by way of limitation, the antibiotic can be administered at an amount of about 1 mg/kg to about 10 mg/kg, about 2 mg/kg to about 10 mg/kg, about 1 mg/kg to about 20 mg/kg, about 2 mg/kg to about 20 mg/kg, about 5 mg/kg to about 10 mg/kg, about 5 mg/kg to about 20 mg/kg, about 5 mg/kg to about 30 mg/kg, about 10 mg/kg to about 20 mg/kg, about 10 mg/kg to about 30 mg/kg, about 10 mg/kg to about 40 mg/kg, about 10 mg/kg to about 50 mg/kg, about 20 mg/kg to about 50 mg/kg, about 30 mg/kg to about 50 mg/kg, about 20 mg/kg to about 60 mg/kg, about 20 mg/kg to about 70 mg/kg, about 20 mg/kg to about 80 mg/kg, about 30 mg/kg to about 60 mg/kg, about 30 mg/kg to about 70 mg/kg, about 30 mg/kg to about 80 mg/kg, about 40 mg/kg to about 60 mg/kg, about 40 mg/kg to about 70 mg/kg, about 40 mg/kg to about 80 mg/kg or about 50 mg/kg to about 80 mg/kg. In certain embodiments, the antibiotic is administered at an amount of about 20 mg/kg to about 50 mg/kg, e.g., about 30 mg/kg. In certain embodiments, the antibiotic is administered at an amount of about 5 mg/kg to about 20 mg/kg, e.g., about 10 mg/kg.

In certain embodiments, the antibiotic can be administered to an animal thrice every day, twice every day, once every day, once every two days, once every three days, once every four days, once every five days, once every six days, once a week, once every two weeks, once every three weeks, or once every month. In certain embodiments, the antibiotic can be administered to an animal one or more times per day. For example, and not by way of limitation, the antibiotic can be administered to an animal once, twice, three, four, five, or more times a day.

In certain embodiments, the presently disclosed methods of treatment can include the identification of the subject having E. ewingii infection. In certain embodiments, the identification is carried out using one of the methods of detection described in Section 3 above.

### 7. Non-Limiting Exemplary Embodiments

In certain non-limiting embodiments, the present disclosure provides a fusion protein comprising the amino acid sequence set forth in SEQ ID NO: 12.

In certain non-limiting embodiments, the present disclosure provides a composition comprising a first fusion protein and a second fusion protein. In certain embodiments, the first fusion protein comprises the amino acid sequence set forth in SEQ ID NO: 12. In certain embodiments, the second fusion protein comprises the amino acid sequence set forth in SEQ ID NO: 16 or SEQ ID NO: 17.

In certain non-limiting embodiments, the present disclosure provides a composition comprising a first fusion protein, a second fusion protein, and a third fusion protein. In certain embodiments, the first fusion protein comprises the amino acid sequence set forth in SEQ ID NO: 12. In certain embodiments, the second fusion protein comprises the amino acid sequence set forth in SEQ ID NO: 16. In certain embodiments, the third fusion protein comprises the amino acid sequence set forth in SEQ ID NO: 17.

In certain non-limiting embodiments, the present disclosure provides a solid phase support comprising a fusion protein comprising the amino acid sequence set forth in SEQ ID NO: 12.

In certain non-limiting embodiments, the present disclosure provides a solid phase support comprising a first fusion protein and a second fusion protein. In certain embodiments, the first fusion protein comprises the amino acid sequence set forth in SEQ ID NO: 12. In certain embodiments, the second fusion protein comprises the amino acid sequence set forth in SEQ ID NO: 16 or SEQ ID NO: 17.

In certain non-limiting embodiments, the present disclosure provides a solid phase support comprising a first fusion protein, a second fusion protein, and a third fusion protein. In certain embodiments, the first fusion protein comprises the amino acid sequence set forth in SEQ ID NO: 12. In certain embodiments, the second fusion protein comprises the amino acid sequence set forth in SEQ ID NO: 16. In certain embodiments, the third fusion protein comprises the amino acid sequence set forth in SEQ ID NO: 17.

### EXAMPLES

The presently disclosed subject matter will be better understood by reference to the following Examples, which are provided as exemplary of the invention, and not by way of limitation.

### Example 1

Ehrlichia spp. are responsible for several emerging zoonoses and capable of causing significant illness in dogs with a potentially fatal rickettsial disease. Accurate diagnostic assays are needed in order to determine appropriate therapy, risk assessment, and treatment monitoring.

The objectives of the present example were to evaluate the performance of the newly developed Accuplex® BioCD automated fluorescence system for the detection of antibodies against E. canis, E. chaffeensis, and E. ewingii in canine serum and compare assay sensitivity and specificity to some commercial tests. The ability to detect antibodies against Ehrlichia pathogens in dogs by the Accuplex automated system was compared with an indirect fluorescent antibody assay (IFA), MFIA (Luminex), SNAP assays, as well as ELISA. Canine blood samples were also assayed for pathogen DNA by polymerase chain reaction (PCR).

The results of the present example demonstrate that the new Accuplex® BioCD Ehrlichia assay is more sensitive than the previous version of the test, as well as alternative commercially available immunoassays. The sensitivity and specificity of the Accuplex® Ehrlichia test are 92% and 95%, respectively, as calculated based on tested clinical canine serum samples. Accuplex® BioCD platform allows multiplexing enhanced Ehrlichia markers in order to achieve improved results. Results of field evaluations show that this test can be reliably used under typical clinical and test conditions to identify dogs exposed to the pathogens of interest.

### Introduction

The Ehrlichiae are a group of gram-negative, obligate intracellular cocci that infect different blood cells in many animal species, as well as in humans. Although these pathogens share phylogenetic similarities, there are multiple differences from tick vectors to host-cell tropism and clinical manifestations. Dogs are susceptible to infection with multiple Ehrlichia spp.; among those are E. canis, transmitted by Rhipicephalus sanguineus (brown dog tick), and E. chaffeensis and E. ewingii, whose primary vector is Amblyomma americanum (lone star tick) (Beall M J, 2012). Coinfections with multiple ehrlichial agents have been reported in dogs (Kordick, 1999). The lone star tick is widely distributed in the eastern, southeastern, and south-central United States, and the brown dog tick is found worldwide (CDC.gov).

In dogs, two leukotrophic diseases are caused by bacteria in the genus Ehrlichia: Canine Monocytic Ehrlichiosis (CME) mainly caused by E. canis and E. chaffeensis, and Canine Granulocytic Ehrlichiosis (CGE), caused by E. ewingii (Aziz, 2023). In recent decades, enhanced and modified transmission patterns of all vector-borne pathogens have been recorded around the globe (Dantas-Torres, 2015) due to multiple factors, including an increase in human population, deforestation, as well as frequent transport of pet animals from one continent to another.

A variety of serologic tests, such as indirect fluorescent antibody (IFA), Western Blot, and ELISAs have been used to verify past or current infections. IFA is the reference standard serologic test for the diagnosis of Ehrlichiosis (CDC.gov) and is being widely used testing method due to its simplicity and cost-effectiveness compared to other approaches.

However, shortcomings of the IFA test are antigenic cross-reactivity with other closely related species that infect dogs, the nature of antigens used in the tests (Stillman B A, 2014), as well as subjective endpoint interpretations and lack of standardization between laboratories (Luo, T., 2010). Since E. ewingii has not been yet cultured, IFAs for antibodies against other Ehrlichia spp. have been used for anti-E. ewingii antibodies detection (Yabsley M J, 2011). Examination of a peripheral blood smear to identify thrombocytopenia and to visually inspect cells for morula is often the only diagnostic test performed prior to treatment (Cohn, L A, 2012).

Identification and differentiation of Ehrlichia and related species with PCR testing is useful in dogs with subclinical & persistent infections, yet some dogs might be PCR negative if the organism is sequestered or persists at undetectable levels, and not so helpful after antibiotic therapy is initiated (Luo, T, 2010). According to the CDC, “a negative PCR result does not rule out the diagnosis, and treatment should not be withheld due to a negative result” (CDC.gov).

In the present example, a newly developed sensitive and reliable immunodiagnostic set of antigens for E. canis, E. chaffeensis, and E. ewingii infection detection were evaluated using the Accuplex® BioCD multiplex system. The presently disclosed Accuplex® advances in characterization of Ehrlichia spp. immune responses have provided new possibilities to significantly improve immunodiagnostics for ehrlichiosis.

### Material and Methods

Study design and samples. Dog blood and serum samples were collected at VCA veterinary clinics and practices. After blood was collected, 1.5 ml was placed into an ethylene diamine tetra-acetic acid (EDTA) tube and then 0.25 ml was pipetted into a 1.5 Eppendorf tube and stored at −80° C. until evaluated for DNA by proprietary PCR assay. The remaining blood was placed into a tube without anticoagulant, allowed to clot, centrifuged at 1,500×g for 10 min, and the sera stored in multiple aliquots at −80° C. until assayed. On the day of collection, the blood samples in EDTA were shipped on cold packs by overnight express to a commercial laboratory for the performance of proprietary PCR assays that amplify the DNA of A. phagocytophilum, A. platys, E. canis, Ehrlichia chaffeensis, and Ehrlichia ewingii using the standard operating procedures of the laboratory (Antech Laboratories, Lake Success, NY). Sera samples were ultimately assayed for Ehrlichia antibodies by IFA, SNAP®4DX®Plus, and Lab 4Dx (4Dx ELISA), IDEXX laboratories assays from commercial manufacturers, following the manufacturer's guidelines, as well as by MFIA (BioVet) and Accuplex® assay.

Accuplex® BioCD System. Accuplex® BioCD, an automated multiplex silicon-wafer-based system, is capable of testing for antigens and antibodies against multiple antigens using small volumes of serum. Additional information on the Accuplex® BioCD system can be found in U.S. Pat. No. 10,100,092, the contents of which are incorporated by reference in their entirety. All markers for multiplexed arrays were printed on high throughput production sciFLEXARRAYER. These assays can be very beneficial in service laboratories because the automated system can lessen inter-assay variability and large numbers of samples can be assayed concurrently. Multiple proprietary peptides were printed on BioCD wafers with the sciFLEXARRAYER S100 system, Scienion. The concentration was determined by assessing the signal: noise with varying antigen concentrations and buffer compositions. Wafers with printed peptides are further processed as described previously (Moroff et al, Vet J., 2014). The reference methods for the detection of antibodies that specifically bind to the amino acid sequences PanEhrl, Mod1, and CanChim were Lab 4Dx Plus Test (7244), SNAP®4Dx®Plus, IFA, and MFIA, comparing data from multiple technological platforms.

IFA tests. Commercially available IFA tests were purchased from VMRD and Fuller laboratories. The assay was performed according to the manufacturer's recommendations. Slides were examined using a UV microscope with filters for fluorescein. To determine the cross-reactivity of samples, IFA tests were performed not only with E. canis and E. chaffeensis kits but also with A. Phagocytophilum slides.

MFIA. Multiplexed fluorometric immunoassays (MFIA), based on Luminex xMAP technology, were performed utilizing beads coated with the same set of antigens as in the presently disclosed Accupex® assay. A suspension of beads coupled with Ehrlichia spp. and Anaplasma recombinant proteins mixed at a concentration of 5×10⁴ beads/ml per bead subset in an assay buffer A (PBS 0.1M, 1% BSA, 5% FBS, NaCl 0.15M). 50 μl of the bead suspension was added to white round-bottom 96 well plates (Corning, Kennebunk, ME, USA) with 50 μl of dog serum diluted in assay buffer A (final serum dilution 1:200). All incubations were performed at room temperature in the dark on a rotating shaker (Compact digital microplate shaker ThermoFisher). After every incubation, the plate was washed twice with washing buffer (PBS, 0.02% Tween 20, 0.1% BSA). Plates were incubated for 60 min and washed. Next, 100 μl of biotin-conjugated rabbit anti-dog IgG (304-065-003 Jackson ImmunoResearch) was added per well in dilution buffer B (1 μg/ml in PBS, 1% BSA) and incubated for 30 min. 100 μl/well of Streptavidin R-Phycoerythrin (5866 Invitrogen) was added at a final concentration of 2 μg/ml in assay buffer B and incubated for 30 min. The beads were then resuspended in a washing buffer and results were read out in a FLEX MAP dispositive (Luminex).

The signal was measured as the median fluorescence intensity (MFI) of at least 50 events of each bead region.

Antigens. All expression constructs were generated by GenScript, NJ, USA; antigens were expressed and purified as described earlier (Moroff et al, 2015). MOD1 (E. ewingii detection) is set forth as SEQ ID NO: 12, CanChimera (Ehrlichia) is set forth as SEQ ID NO: 16, and PanEhrlichia (Ehrlichia) is set forth as SEQ ID NO: 17. SEQ ID Nos: 12, 16, and 17 are reproduced below.

[SEQ ID NO: 12]
SNIGAETKKTFGLEVSYDGAKIEDGGAETIATFGLSKTYNGAQIT
DGGAETKKVLGLNKNYDGAKIEDGAETKRTFGLDKNEGLTDGGAE
TKNSIALEKNYDGAKIEDGGAETIATFGLSKTYNGAQITDGAETS
KKFVLENNYDGAKIEDGGAETKRTFGLDIGYFGAKIEDGAETKKT
FGLESFYEGAKIEDGAETKKTFGLEGSYEGAKIEDGAETIATFGL
SKTYNGAQITD
[SEQ ID NO: 16]
SNIGTEDSVSAPATEDSVSAPATEDSVSAPATEDSVSAPATEDSV
SAPATEDSVSAPAGGGSYNHNTGLLDLDSDILNMLYSYNHNTGLL
DLDSDILNMLYSYNHNTGLLDLDSDILNMLYGGGSKEESTPEVKA
EDLQPAVDSKEESTPEVKAEDLQPAVDSKEESTPEVKAEDLQPAV
D
[SEQ ID NO: 17]
SNIGNTTTGVFGLKQDWDGATIKDGGNTTVGVFGLKQNWDGSAIS
NGGNPTVALYGLKQDWNGVSAGGNTTVGVFGIEQDWDRCVISGGN
PTVALYGLKQDWEGISSGGKSTVGVFGLKHDWDGSPILKGGNTTT
GVFGLKQDWDGSTISGGNTTTGVFGLKQDWDGATIKDGGNPTVAL
YGLKQDWNGVSAGGKSTVGVFGLKHDWDGSPILKGGNTTVGVFGI
EQDWDRCVISGGNTTTGVFGLKQDWDGATIKDGGNTTVGVFGLKQ
NWDGSAISNGGKSTVGVFGLKHDWDGSPILK

### Results

Clinical samples of dogs were tested for E. canis, E. chaffeensis, and E. ewingii infections using conventional assays such as IFA, SNAP®4Dx®Plus, Lab4Dx tests (IDEXX Laboratories), as well as MFIA (BioVet), and the presently disclosed multiplex Ehrlichia assay (ACCUPLEX®, Antech). Three (3) sets of samples were evaluated: a) a set of 155 clinical samples from New York reference laboratories; b) 38 clinical samples confirmed with a proprietary qPCR; and c) 32 samples selected in collaboration with Colorado State University (CSU). Table 1 shows a data comparison of tested canine serum samples using the presently disclosed Accuplex®, SNAP®4Dx®, MFIA, and IFA. Table 1 is provided below.

After the analysis of all the samples, 60 samples were determined as positive with SNAP®4Dx®Plus, and 72 samples, 71 samples, and 80 samples were determined as positive with the presently disclosed Accuplex® set of antigens, MFIA, and IFA, respectively.

Sensitivity and specificity for the presently disclosed Accuplex® were 93.3% and 83.2%, respectively, without consideration of IFA confirmatory test results (Table 2). 16 serum samples that were Accuplex® positive and SNAP®4Dx® negative were further confirmed as positive with IFA, bringing the specificity of the presently disclosed Accuplex® to 97.53%. Table 2 is provided below.

The results of 155 clinical sample tests show that the presently disclosed Accuplex® is capable of reliably detecting Ehrlichia spp. in canine serum. 147 samples were tested, and their results were compared on the presently disclosed Accuplex® and Lab4Dx platforms (8 samples did not have sufficient volume for submission). Responses from 147 samples generated 58 positive results (39%) with 4Dx®ELISA, 64 positives (44%) on the presently disclosed Accuplex® platform, and 65 (44%) and 73 (50%) with MFIA and IFA respectively. Table 3A shows Ehrlichia spp. detection with the presently disclosed Accuplex® and Lab4Dx® for 147 canine clinical samples. Table 3B shows Accuplex® and Lab4Dx performance comparison. Tables 3A and 3B are provided below.

Statistical analysis of the sensitivity and specificity of the presently disclosed Accuplex® and 4Dx®ELISA was done considering a potential cross-reactivity between Ehrlichia spp. and Anaplasma spp, particularly when the pathogens were present at very high titers (Sainz, A 2015). Similar cross-reactivity observations were stated in the IFA kit performance characteristics document (Fuller laboratory).

Sensitivity and specificity for the newly developed Ehrlichia Accuplex® enhancement markers were 86% and 96% when Anaplasma spp cross-reactivity was not considered, and 92% and 95%, respectively, with the consideration of such possibility. The sensitivity and specificity of reference Lab4Dx® Ehrlichia calculated with the consideration of the Anaplasma factor was 77% and 98%, respectively.

Statistically calculated comparison between two assay platforms shows strong overall agreement (0.8-0.9), with negative and positive agreements being 0.87 and 0.9, respectively.

To evaluate the performance of MOD1 antigen in detecting and differentiating E. ewingii, a set of 32 samples, selected and confirmed by serology and PCR at CSU, was tested on the BioCD platform in a blind mode. Table 4A shows the results. Table 4B shows the summary of the same. Tables 4A and 4B are provided below.

32 samples were analyzed at CSU by qPCR and serology before the presently disclosed Accuplex®, MFIA, and SNAP®4Dx®Plus testing, with 16 samples confirmed as E. ewingii positive. All 16 E. ewingii positive samples were detected by the presently disclosed Accuplex® markers, and 13 samples were detected by SNAP®4Dx®Plus. Sensitivity and specificity for the presently disclosed Accuplex® was 100% for this set, with SNAP®4Dx®Plus sensitivity/specificity at 81.2% and 100%, accordingly. The presently disclosed Accuplex® platform detected all positive E. ewingii samples in this set. All positive sequencing results confirmed E. ewingii sequences for the samples identified as such by Accuplex®. Table 5A shows Accuplex® results on Ehrlichia spp. detection for qPCR-confirmed samples. Table 5B shows a summary of results of Table 5A. Tables 5A and 5B are provided below.

38 canine DNA samples were tested with a proprietary qPCR system (Antech) to determine Ehrlichia spp. and/or Anaplasma Spp. 34 samples were qPCR Ehrlichia spp. positive (E. canis and E. ewingii) and 4 samples were positive for A. phagocytophilum. Corresponding serum samples were analyzed with the presently disclosed Accuplex®, MFIA, and SNAP®4Dx®Plus. Accuplex® and MFIA positively identified 33 out of 34 qPCR-confirmed samples with one sample not having sufficient volume for testing on Accuplex®. Genotype reports of the tested samples clearly show that presently disclosed Accuplex® markers are capable of detecting Ehrlichia spp. of various genotypes, i.e., Columbia, Turkey, and Thailand strains among others.

Worldwide distribution of E. canis and other Ehrlichia spp. has been reported in an increasing number of publications (Aguiar, 2008, Sainz A. 2015). The combination of PCR detection with an immunodiagnostic approach significantly improves early diagnosis in the acute stage of infection with better sensitivity, providing the best evidence of recent infection, helping with result interpretation, and treatment selection and ensuring better prognosis. Correct and timely testing and reporting of vector-borne infections are important to improve understanding of evolving trends, the geographical distribution of these diseases, and how common these infections are.

### SUMMARY

Conventional assays used to diagnose ehrlichiosis, such as IFA, have limitations related to the nature of antigens used in the tests. Because E. ewingii has not been successfully cultured, IFAs for antibodies against other Ehrlichia spp. (E. canis and E. chaffeensis) have been widely used for detecting anti-E. ewingii antibodies in dogs. Serologic cross-reactivity among E. canis, E. ewingii, and E. chaffeensis has been reported, as a result, diagnosis on the basis of whole-cell ELISA or IFA results is limited (Stillman et al, 2014). In the present example, it was evaluated Accuplex® assay performance developed with novel species-specific recombinant antigens. It has been determined that the sensitivity and specificity of the presently disclosed Accuplex® Ehrlichia assay are 92% and 95%, respectively.

### Tables

TABLE 1
Accuplex ®, SNAP ® 4Dx ®, MFIA, and IFA
data comparison of 155 canine serum samples.

| | SNAP ® 4Dx ® Plus | | Accuplex ® | MFIA | |
|---|---|---|---|---|---|
| Sample ID | Ehrlichia | GP36 | Ehrlichia | Ehrlichia | IFA |
| 1 | Neg | neg | Neg | Neg | pos |
| 2 | Neg | neg | Neg | Neg | pos |
| 3 | Neg | neg | Pos | Pos | pos |
| 4 | Pos | Pos | Pos | Pos | pos |
| 5 | Pos | neg | Pos | Pos | pos |
| 6 | Neg | neg | Neg | Neg | neg |
| 7 | Neg | neg | Neg | Neg | neg |
| 8 | Neg | neg | Neg | Neg | neg |
| 9 | Neg | neg | Neg | Neg | neg |
| 10 | Neg | neg | Neg | Neg | neg |
| 11 | Neg | neg | Neg | Neg | neg |
| 12 | Neg | neg | Neg | Neg | neg |
| 13 | Neg | neg | Neg | Neg | neg |
| 14 | Neg | neg | Neg | Neg | neg |
| 15 | Pos | pos | Pos | Pos | pos |
| 16 | Neg | neg | Neg | Neg | neg |
| 17 | Neg | neg | Pos | BDL | pos |
| 18 | Neg | neg | Neg | Neg | neg |
| 19 | Neg | neg | Neg | Neg | neg |
| 20 | Neg | neg | Neg | Neg | neg |
| 21 | Neg | neg | Neg | Neg | neg |
| 22 | Neg | neg | Neg | Neg | neg |
| 23 | Neg | neg | Neg | Neg | neg |
| 24 | Neg | neg | Neg | Neg | pos |
| 25 | Neg | neg | Pos | Pos | pos |
| 26 | Neg | neg | Pos | Pos | pos |
| 27 | Neg | neg | Neg | Neg | neg |
| 28 | Pos | pos | Pos | Pos | pos |
| 29 | Pos | pos | Pos | Pos | pos |
| 30 | Pos | pos | Pos | Pos | pos |
| 31 | Pos | pos | Pos | Pos | pos |
| 32 | Neg | pos | Pos | Pos | pos |
| 33 | Neg | neg | Pos | Neg | pos |
| 34 | Neg | neg | Neg | Neg | pos |
| 35 | Neg | pos | Neg | Neg | pos |
| 36 | Neg | neg | Pos | Pos | pos |
| 37 | Neg | neg | Neg | Neg | neg |
| 38 | Neg | neg | Neg | Neg | neg |
| 39 | Neg | neg | Neg | Neg | neg |
| 40 | Neg | neg | Neg | Neg | neg |
| 41 | Neg | neg | Neg | Neg | neg |
| 42 | Neg | neg | Neg | Neg | neg |
| 43 | Neg | neg | Neg | Neg | neg |
| 44 | Neg | neg | Neg | Neg | neg |
| 45 | Neg | pos | Pos | Pos | pos |
| 46 | Neg | pos | Pos | Pos | pos |
| 47 | Pos | neg | Pos | Pos | pos |
| 48 | Neg | neg | Neg | Neg | neg |
| 49 | Neg | neg | Neg | Neg | neg |
| 50 | Neg | pos | Pos | Pos | pos |
| 51 | Neg | neg | Neg | Neg | neg |
| 52 | Neg | neg | Neg | Neg | neg |
| 53 | Pos | neg | Pos | Pos | pos |
| 54 | Neg | neg | Neg | Neg | neg |
| 55 | Neg | neg | Neg | Neg | neg |
| 56 | Neg | neg | Neg | Neg | neg |
| 57 | Neg | neg | Neg | Neg | neg |
| 58 | Neg | neg | Neg | Neg | neg |
| 59 | Neg | neg | Neg | Neg | neg |
| 60 | Neg | neg | Neg | Neg | neg |
| 61 | Neg | neg | Neg | Neg | neg |
| 62 | Pos | neg | Pos | Pos | pos |
| 63 | Neg | neg | Neg | Neg | neg |
| 64 | Neg | neg | Neg | Neg | neg |
| 65 | Neg | neg | Neg | Neg | neg |
| 66 | Neg | neg | Neg | Neg | neg |
| 67 | Neg | neg | Neg | Neg | neg |
| 68 | Neg | pos | Pos | Pos | neg |
| 69 | Pos | pos | Pos | Pos | pos |
| 70 | Neg | neg | Pos | Neg | pos |
| 71 | Neg | pos | Pos | Pos | pos |
| 72 | Neg | neg | Neg | Neg | neg |
| 73 | Pos | pos | Pos | Pos | pos |
| 74 | Neg | neg | Neg | Neg | neg |
| 75 | Neg | neg | Neg | Neg | neg |
| 76 | Pos | pos | Pos | Pos | pos |
| 77 | Pos | pos | Pos | Pos | pos |
| 78 | Neg | neg | Neg | Neg | pos |
| 79 | Pos | pos | Pos | Pos | pos |
| 80 | Pos | neg | Pos | Pos | pos |
| 81 | Pos | pos | Pos | Pos | pos |
| 82 | Neg | pos | Pos | Neg | pos |
| 83 | Pos | pos | Pos | Pos | pos |
| 84 | Neg | neg | Neg | Neg | neg |
| 85 | Neg | neg | Neg | Neg | neg |
| 86 | Neg | neg | Pos | Pos | neg |
| 87 | Pos | neg | Pos | Pos | pos |
| 88 | Neg | neg | Pos | Pos | pos |
| 89 | Neg | neg | Neg | Neg | neg |
| 90 | Neg | neg | Neg | Neg | neg |
| 91 | Neg | neg | Neg | Neg | neg |
| 92 | Neg | neg | Neg | Neg | neg |
| 93 | Neg | neg | Neg | Neg | neg |
| 94 | Neg | neg | Neg | Neg | neg |
| 95 | Neg | neg | Neg | Neg | neg |
| 96 | Neg | neg | Neg | Neg | neg |
| 97 | Neg | neg | Neg | Neg | neg |
| 98 | Neg | neg | Neg | Neg | neg |
| 99 | Neg | neg | Neg | Neg | neg |
| 100 | Neg | neg | Neg | Neg | neg |
| 101 | Neg | neg | Neg | Pos | neg |
| 102 | Neg | neg | Neg | Pos | pos |
| 103 | Neg | neg | Neg | Neg | neg |
| 104 | Neg | neg | Neg | Neg | neg |
| 105 | Neg | neg | Neg | Neg | neg |
| 106 | Neg | neg | Neg | Neg | neg |
| 107 | Neg | neg | Neg | Neg | neg |
| 108 | Neg | neg | Neg | Neg | neg |
| 109 | Neg | neg | Neg | Neg | neg |
| 110 | Neg | neg | Neg | Neg | neg |
| 111 | Neg | neg | Neg | Neg | neg |
| 112 | Pos | neg | Pos | Pos | pos |
| 113 | Pos | neg | Pos | Pos | pos |
| 114 | Pos | pos | Pos | Pos | pos |
| 115 | Pos | pos | Pos | Pos | pos |
| 116 | Pos | neg | Neg | Neg | pos |
| 117 | Pos | neg | Pos | Pos | pos |
| 118 | Pos | pos | Pos | Pos | pos |
| 119 | Pos | pos | Pos | Pos | pos |
| 120 | Pos | pos | Pos | Pos | pos |
| 121 | Pos | pos | Pos | Pos | pos |
| 122 | Neg | neg | Neg | Neg | neg |
| 123 | Pos | pos | Pos | Pos | pos |
| 124 | Pos | pos | Pos | Pos | pos |
| 125 | Pos | 6363 | Pos | Pos | pos |
| 126 | Pos | pos | Pos | Pos | pos |
| 127 | Pos | neg | Pos | Pos | pos |
| 128 | Pos | pos | Pos | Pos | pos |
| 129 | Pos | neg | Neg | Neg | pos |
| 130 | Neg | neg | Neg | Neg | neg |
| 131 | Pos | neg | Neg | Pos | pos |
| 132 | Pos | neg | Pos | Pos | pos |
| 133 | Pos | pos | Pos | Pos | pos |
| 134 | Pos | neg | Pos | Pos | pos |
| 135 | Pos | pos | Pos | Pos | pos |
| 136 | Pos | neg | Pos | Pos | pos |
| 137 | Pos | neg | Pos | Pos | pos |
| 138 | Pos | neg | Pos | Pos | pos |
| 139 | Pos | neg | Neg | Neg | pos |
| 140 | Pos | neg | Pos | Pos | pos |
| 141 | Pos | neg | Pos | Pos | pos |
| 142 | Pos | pos | Pos | Pos | pos |
| 143 | Pos | neg | Pos | Pos | pos |
| 144 | Neg | neg | Neg | Neg | neg |
| 145 | Pos | pos | Pos | Pos | pos |
| 146 | Pos | neg | Pos | Pos | pos |
| 147 | Pos | pos | Pos | Pos | pos |
| 148 | Pos | pos | Pos | Pos | pos |
| 149 | Pos | pos | Pos | Pos | pos |
| 150 | Pos | neg | Pos | Pos | neg |
| 151 | Pos | pos | Pos | Pos | pos |
| 152 | Pos | neg | Pos | Pos | pos |
| 153 | Pos | pos | Pos | Pos | pos |
| 154 | Pos | pos | Pos | Pos | pos |
| SAMPOOL00582 | Pos | pos | Pos | Pos | pos |
| Positives | 60 | 42 | 72 | 71 | 80 |

TABLE 2
Sensitivity and Specificity analysis for the set of 155 canine serum samples:

| | | Accuplex ® | | | | | |
|---|---|---|---|---|---|---|---|
| | | Ehrlichia | | | | | |
| | | POS | NEG | | | SNAP ®4Dx ®Plus | Accuplex ® |
| SNAP ®4Dx ®Plus, | POS | 56 | 4 | 60 | Sensitivity | 77.78 | 93.3 |
| Ehrlichia | NEG | 16 | 79 | 95 | Specificity | 95.18 | 83.2 |
| | | 72 | 83 | 155 | | | |

With IFA data, Accuplex ® Specificity: 97.53%
TN 79
FP: out of 16 samples, 14 were confirmed with ELISA or IFA.
FP 2

TABLE 3A
Ehrlichia spp. detection with Accuplex ® and Lab 4Dx ® (4Dx ELISA)

| | Ehrlichia 4Dx | | | |
|---|---|---|---|---|
| Animal ID | ELISA | ACCUPLEX ® EHRLICHIA | IFA (Ehrlichia) | MFIA |
| 1 | Neg | Neg | pos | Neg |
| 2 | Neg | Neg | pos | Neg |
| 3 | Neg | Pos | pos | Pos |
| 4 | Pos | Pos | pos | Pos |
| 5 | Pos | Pos | pos | Pos |
| 6 | Neg | Neg | Neg | Neg |
| 7 | Neg | Neg | Neg | Neg |
| 8 | Neg | Neg | Neg | Neg |
| 9 | Neg | Neg | Neg | Neg |
| 10 | Neg | Neg | Neg | Neg |
| 11 | Neg | Neg | Neg | Neg |
| 12 | Neg | Neg | Neg | Neg |
| 13 | Neg | Neg | Neg | Neg |
| 14 | Neg | Neg | Neg | Neg |
| 15 | Pos | Pos | pos | Pos |
| 16 | Neg | Neg | Neg | Neg |
| 17 | Neg | Pos | pos | Neg |
| 18 | Neg | Neg | Neg | Neg |
| 19 | Neg | Neg | Neg | Neg |
| 20 | Neg | Neg | Neg | Neg |
| 21 | Neg | Neg | Neg | Neg |
| 22 | Neg | Neg | Neg | Neg |
| 23 | Neg | Neg | Neg | Neg |
| 24 | Neg | Neg | pos | Neg |
| 25 | Neg | Pos | pos | Pos |
| 26 | Neg | Pos | pos | Pos |
| 27 | Neg | Neg | Neg | Neg |
| 28 | Pos | Pos | pos | Pos |
| 29 | Pos | Pos | pos | Pos |
| 30 | Pos | Pos | pos | Pos |
| 31 | Pos | Pos | pos | Pos |
| 32 | Neg | Pos | pos | Neg |
| 33 | Neg | Neg | pos | Neg |
| 34 | Neg | Neg | pos | Neg |
| 35 | Neg | Neg | Neg | Neg |
| 36 | Neg | Neg | Neg | Neg |
| 37 | Neg | Neg | Neg | Neg |
| 38 | Neg | Neg | Neg | Neg |
| 39 | Neg | Neg | Neg | Neg |
| 40 | Neg | Neg | Neg | Neg |
| 41 | Neg | Neg | Neg | Neg |
| 42 | Neg | Neg | Neg | Neg |
| 43 | Neg | Pos | pos | Pos |
| 44 | Pos | Pos | pos | Pos |
| 45 | Neg | Neg | Neg | Neg |
| 46 | Neg | Neg | Neg | Neg |
| 47 | Neg | Pos | pos | Pos |
| 48 | Neg | Neg | Neg | Neg |
| 49 | Neg | Neg | Neg | Neg |
| 50 | Pos | Pos | pos | Pos |
| 51 | Neg | Neg | Neg | Neg |
| 52 | Neg | Neg | Neg | Neg |
| 53 | Neg | Neg | Neg | Neg |
| 54 | Neg | Neg | Neg | Neg |
| 55 | Neg | Neg | Neg | Neg |
| 56 | Neg | Neg | Neg | Neg |
| 57 | Neg | Neg | Neg | Neg |
| 58 | Neg | Neg | Neg | Neg |
| 59 | Pos | Pos | pos | Pos |
| 60 | Neg | Neg | Neg | Neg |
| 61 | Neg | Neg | Neg | Neg |
| 62 | Neg | Neg | Neg | Neg |
| 63 | Neg | Neg | Neg | Neg |
| 64 | Neg | Neg | Neg | Neg |
| 65 | Neg | Pos | neg | Pos |
| 66 | Pos | Pos | pos | Pos |
| 67 | Neg | Pos | pos | Pos |
| 68 | Neg | Pos | pos | Pos |
| 69 | Neg | Neg | Neg | Neg |
| 70 | Neg | Neg | Neg | Neg |
| 71 | Neg | Neg | Neg | Neg |
| 72 | Pos | Pos | pos | Pos |
| 73 | Neg | Neg | pos | Neg |
| 74 | Pos | Pos | pos | Pos |
| 75 | Pos | Pos | pos | Pos |
| 76 | Pos | Pos | pos | Pos |
| 77 | Neg | Pos | pos | Pos |
| 78 | Pos | Pos | pos | Pos |
| 79 | Neg | Neg | Neg | Neg |
| 80 | Neg | Neg | Neg | Neg |
| 81 | Pos | Pos | pos | Pos |
| 82 | Neg | Pos | pos | Pos |
| 83 | Neg | Neg | Neg | Neg |
| 84 | Neg | Neg | Neg | Neg |
| 85 | Neg | Neg | Neg | Neg |
| 86 | Neg | Neg | Neg | Neg |
| 87 | Pos | Neg | Neg | Neg |
| 88 | Neg | Neg | Neg | Neg |
| 89 | Neg | Neg | Neg | Neg |
| 90 | Neg | Neg | Neg | Neg |
| 91 | Neg | Neg | Neg | Neg |
| 92 | Neg | Neg | Neg | Neg |
| 93 | Neg | Neg | Neg | Neg |
| 94 | Neg | Neg | Neg | Neg |
| 95 | Neg | Neg | Neg | Pos |
| 96 | Neg | Neg | pos | Pos |
| 97 | Neg | Neg | Neg | Neg |
| 98 | Neg | Neg | Neg | Neg |
| 99 | Neg | Neg | Neg | Neg |
| 100 | Neg | Neg | Neg | Neg |
| 101 | Neg | Neg | Neg | Neg |
| 102 | Neg | Neg | Neg | Neg |
| 103 | Neg | Neg | Neg | Neg |
| 104 | Neg | Neg | Neg | Neg |
| 105 | Neg | Neg | Neg | Neg |
| 106 | Pos | Pos | pos | Pos |
| 107 | Pos | Pos | pos | Pos |
| 108 | Pos | Pos | pos | Pos |
| 109 | Pos | Pos | pos | Pos |
| 110 | Neg | Neg | pos | Neg |
| 111 | Pos | Pos | pos | Pos |
| 112 | Pos | Pos | pos | Pos |
| 113 | Pos | Pos | pos | Pos |
| 114 | Pos | Pos | pos | Pos |
| 115 | Pos | Pos | pos | Pos |
| 116 | Pos | Neg | Neg | Neg |
| 117 | Pos | Pos | pos | Pos |
| 118 | Pos | Pos | pos | Pos |
| 119 | Pos | Pos | pos | Pos |
| 120 | Pos | Pos | pos | Pos |
| 121 | Pos | Pos | pos | Pos |
| 122 | Pos | Pos | pos | Pos |
| 123 | Pos | Neg | pos | Neg |
| 124 | Pos | Neg | Neg | Neg |
| 125 | Pos | Neg | pos | Pos |
| 126 | Pos | Pos | pos | Pos |
| 127 | Pos | Pos | pos | Pos |
| 128 | Pos | Pos | pos | Pos |
| 129 | Pos | Pos | pos | Pos |
| 130 | Pos | Pos | pos | Pos |
| 131 | Pos | Pos | pos | Pos |
| 132 | Pos | Neg | pos | Neg |
| 133 | Pos | Pos | pos | Pos |
| 134 | Pos | Pos | pos | Pos |
| 135 | Pos | Pos | pos | Pos |
| 136 | Pos | Pos | pos | Pos |
| 137 | Neg | Neg | Neg | Neg |
| 138 | Pos | Pos | pos | Pos |
| 139 | Pos | Pos | pos | Pos |
| 140 | Pos | Pos | pos | Pos |
| 141 | Pos | Pos | pos | Pos |
| 142 | Pos | Pos | Neg | Pos |
| 143 | Pos | Pos | pos | Pos |
| 144 | Pos | Pos | pos | Pos |
| 145 | Pos | Pos | pos | Pos |
| 146 | Pos | Pos | pos | Pos |
| 147 | Pos | Pos | pos | Pos |
| 147 | 58 | 64 | 73 | 65 |
| | 39% | 44% | 50% | 44% |

TABLE 3B

| | Ehrlichia 4Dx ® | ACCUPLEX ® | IFA | |
|---|---|---|---|---|
| Animal ID | ELISA | EHRLICHIA | (Ehrlichia) | MFIA |
| 147 | 58 | 64 | 73 | 65 |
| | 39% | 44% | 50% | 44% |

TABLE 4A
Accuplex ® results on detecting and differentiating E. ewingii.

| | SNAP ® | | MFIA | | Accuplex ® | | | |
|---|---|---|---|---|---|---|---|---|
| | 4Dx ® | 4Dx ® | | Other | | Other | | |
| | Plus | Plus | | Positive | E. ewingii | Positive | qPCR | |
| ID | Ehrlichia | Anap1 | E. ewingii | responses | result | responses | E. ewingii | Sequencing |
| 1 | Pos | neg | POS | | POS | | POS | E. ewingii |
| | | | | | | | | EF116932.1 |
| 2 | neg | neg | neg | | neg | | — | Ewingii |
| | | | | | | | | sequencing - no |
| | | | | | | | | ID |
| 3 | Pos | neg | POS | | POS | | POS | |
| 4 | neg | neg | neg | | neg | | — | Ewingii |
| | | | | | | | | sequencing - no |
| | | | | | | | | ID |
| 5 | neg | neg | neg | | neg | | — | Ewingii |
| | | | | | | | | sequencing - no |
| | | | | | | | | ID |
| 6 | neg | neg | neg | | neg | | — | Ewingii |
| | | | | | | | | sequencing - no |
| | | | | | | | | ID |
| 7 | neg | neg | neg | | neg | | — | Ewingii |
| | | | | | | | | sequencing - no |
| | | | | | | | | ID |
| 8 | Pos | neg | POS | | POS | | POS | E. ewingii |
| | | | | | | | | EF116932.1 |
| 9 | neg | neg | Neg | | POS | | POS | E. ewingii |
| | | | | | | | | AF287962.1 |
| 10 | neg | neg | Neg | | neg | | — | |
| 11 | Pos | neg | POS | E. | POS | E. chaffeensis | POS | |
| | | | | chaffeensis | | | | |
| 12 | Pos | neg | POS | | POS | | POS | |
| 13 | Pos | neg | Neg | E. canis / E. | neg | E. canis / E. | E. canis | E. canis |
| | | | | chaffeensis | | chaffeensis | | Q085429 |
| 14 | neg | neg | POS | | POS | | POS | E. ewingii |
| | | | | | | | | EF116932.1 |
| 15 | neg | neg | Neg | | neg | | — | |
| 16 | neg | neg | POS | | POS | | POS | |
| 17 | Pos | neg | POS | Ehrlichia | POS | | POS | |
| 18 | neg | neg | Neg | | neg | | — | |
| 19 | neg | neg | Neg | | neg | | — | |
| 20 | neg | neg | Neg | | neg | | — | |
| 21 | neg | neg | Neg | | Neg | | — | |
| 22 | Pos | neg | POS | Ehrlichia | POS | | POS | E. ewingii |
| | | | | | | | | EF116932.1 |
| 23 | Pos | neg | POS | | POS | | POS | |
| 24 | Pos | neg | POS | | POS | | — | Ewingii |
| | | | | | | | | sequencing - no |
| | | | | | | | | ID |
| 25 | neg | Pos | Neg | | POS | | — | |
| 26 | Pos | neg | POS | E. canis | POS | E. canis | E. canis | E. canis |
| | | | | | | | & | KT357369 |
| | | | | | | | A. platys, | |
| 27 | Pos | neg | POS | | POS | | POS | |
| 28 | Pos | neg | POS | E. canis | POS | E. canis | E. ewingii | GP36 |
| | | | | | | | & | sequencing - |
| | | | | | | | E. canis | E. Ewingii |
| 29 | neg | neg | Neg | | neg | | — | |
| 30 | neg | neg | Neg | | neg | | — | |
| 31 | neg | neg | Neg | | neg | | | |
| 32 | Neg | neg | Neg | | neg | | — | |
| E. ewingii | NA | | 14 | | 16 | | 13 | |
| Only | | | | | | | | |
| Total | 13 | | 15 | | 17 | | 15 | |
| Ehrlichia | | | | | | | | |
| spp | | | | | | | | |

TABLE 4B

| | Accuplex ® | | SNAP ® | MFIA |
|---|---|---|---|---|
| | E. ewingii | qPCR | 4Dx ® Plus | E. |
| Platform | result | E. ewingii | Ehrlichia | ewingii |
| E. ewingii Only | 16 | 13 | NA | 14 |
| Total Ehrlichia spp | 17 | 15 | 13 | 15 |

TABLE 5A
Accuplex ® results on Ehrlichia spp. detection for qPCR-confirmed samples.

| | | | | | 4Dx ® | | | |
|---|---|---|---|---|---|---|---|---|
| Animal | | Ehrlichia | FV PCR | 4Dx ®Plus | Plus | GP | Accuplex ® | LUMINEX ® |
| ID | FV qPCR result | Genotyping | EHRLICHIA | Anapl | Ehrlichia | 36 | Ehrlichia | Ehrlichia |
| 1 | Anaplasma | no DNA | neg | neg | neg | neg | neg | Neg |
| | phagocytophilum | | | | | | | |
| 2 | Anaplasma | no DNA | neg | neg | neg | neg | neg | Neg |
| | phagocytophilum | | | | | | | |
| 3 | Ehrlichia canis | E. canis | pos | neg | pos | neg | pos | pos |
| | | Columbia | | | | | | |
| 4 | Ehrlichia canis | no DNA | pos | neg | pos | neg | pos | pos |
| 5 | Ehrlichia canis | E. canis Peru | pos | neg | pos | neg | pos | pos |
| 6 | Ehrlichia canis | E. canis | pos | pos | pos | neg | pos | pos |
| | | Thailand | | | | | | |
| 7 | Ehrlichia canis | E. canis | pos | neg | pos | neg | pos | pos |
| | | Columbia | | | | | | |
| 8 | Ehrlichia canis | E. canis | pos | neg | neg | pos | pos | pos |
| | | Columbia | | | | | | |
| 9 | E. ewingii | E. ewingii | pos | neg | neg | neg | pos | pos |
| 10 | E. ewingii | E. ewingii | pos | neg | pos | neg | pos | pos |
| 11 | Ehrlichia canis | no DNA | pos | neg | pos | pos | pos | pos |
| 12 | Ehrlichia canis | E. canis | pos | pos | pos | pos | pos | pos |
| | | Columbia | | | | | | |
| 13 | Ehrlichia canis | * | pos | neg | pos | neg | pos | pos |
| 14 | Co-Infection | E. canis | pos | neg | neg | neg | po | pos |
| | | Columbia | | | | | | |
| 15 | Co-Infection | E. canis | pos | neg | neg | pos | pos | pos |
| | | Turkey | | | | | | |
| 16 | Ehrlichia canis | | pos | neg | pos | neg | pos | pos |
| 17 | Ehrlichia canis | E. canis | pos | neg | pos | neg | pos | pos |
| | | Columbia | | | | | | |
| 18 | Ehrlichia canis | * | pos | neg | pos | pos | pos | pos |
| 19 | Ehrlichia canis | * | pos | neg | pos | neg | pos | pos |
| 20 | Ehrlichia canis | * | pos | neg | neg | neg | pos | pos |
| 21 | Ehrlichia canis | E. canis | pos | pos | pos | NA | NA | pos |
| | | Columbia | | | | | | |
| 22 | Ehrlichia canis | * | pos | pos | pos | neg | pos | pos |
| 23 | Ehrlichia canis | * | pos | pos | pos | pos | pos | pos |
| 24 | Ehrlichia ewingii | | pos | neg | pos | neg | pos | pos |
| 25 | Ehrlichia canis | ** | pos | neg | pos | neg | pos | pos |
| 26 | Ehrlichia canis | E. canis | pos | neg | neg | neg | pos | pos |
| | | Turkey | | | | | | |
| 27 | Ehrlichia canis | E. canis Jake | pos | neg | pos | neg | pos | pos |
| 28 | Ehrlichia canis | E. canis | pos | neg | pos | neg | pos | pos |
| | | Columbia | | | | | | |
| 29 | Ehrlichia canis | * | pos | neg | pos | neg | pos | pos |
| 30 | Ehrlichia canis | no DNA | pos | pos | pos | neg | pos | pos |
| 31 | Ehrlichia canis | E. canis | pos | neg | pos | neg | pos | pos |
| | | Columbia | | | | | | |
| 32 | Anaplasma | no DNA | neg | pos | neg | neg | neg | Neg |
| | phagocytophilum | | | | | | | |
| 33 | Ehrlichia canis | E. canis | pos | neg | pos | neg | pos | pos |
| | | Columbia | | | | | | |
| 34 | Anaplasma | * | neg | neg | pos | neg | pos | pos |
| | phagocytophilum | | | | | | | |
| 35 | Ehrlichia canis | * | pos | neg | pos | neg | pos | pos |
| 36 | Ehrlichia canis | * | pos | neg | neg | neg | pos | pos |
| 37 | Ehrlichia canis | * | pos | neg | pos | neg | pos | pos |
| 38 | Ehrlichia canis | * | pos | neg | neg | neg | pos | Neg |
| | | | 34 | | 27 | 6 | 34 | 34 |

TABLE 5B

| | Ehrlichia | | | | |
|---|---|---|---|---|---|
| Total | FV | SNAP ® | Positives | | |
| Samples | qPCR | 4Dx ® Plus | GP36 | Accuplex ® | MFIA |
| tested | Ehrlichia | Ehrlichia | Ehrlichia | Ehrlichia | Ehrlichia |
| 38 | 34 | 27 | 6 | 34 | 34 |

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A fusion protein comprising at least two polypeptides comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or a combination thereof.

2. The fusion protein of claim 1, comprising an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 10, SEQ ID NO: 11, SEQ ID NO: 12, SEQ ID NO: 13, SEQ ID NO: 14, or SEQ ID NO: 15.

3. The fusion protein of claim 1, comprising an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 12.

4. The fusion protein of claim 3, comprising the amino acid sequence set forth in SEQ ID NO: 12.

5. A nucleic acid molecule comprising a polynucleotide encoding the fusion protein of claim 1.

6. A vector comprising the nucleic acid molecule of claim 5.

7. A host cell comprising the fusion protein of claim 1.

8. A method of producing a fusion protein comprising culturing the host cell of claim 1 in a culture medium and purifying the fusion protein.

9. A composition comprising a fusion protein of claim 1.

10. A solid phase support comprising a fusion protein of claim 1.

11. The solid phase support of claim 10, further comprising a second fusion protein.

12. The solid phase support of claim 11, wherein the second fusion protein comprises the amino acid sequence set forth in SEQ ID NO: 16 or SEQ ID NO: 17.

13. The solid phase support of claim 10, further comprising a second fusion protein and a third fusion protein.

14. The solid phase support of claim 13, wherein a) the second fusion protein comprises the amino acid sequence set forth in SEQ ID NO: 16, and b) the third fusion protein comprises the amino acid sequence set forth in SEQ ID NO: 17.

15. A kit comprising contacting the fusion protein of claim 1.

16. A method of treating and/or delaying progression of E. ewingii infection of a subject in need thereof comprising:
a) identifying the subject as having an E. ewingii infection; and
b) administering an antibiotic to the subject.

17. The method of claim 16, wherein the identifying comprises:
a) contacting the fusion protein comprising an amino acid sequence that is at least about 80%, about 85%, about 90%, about 91%, about 92%, about 93%, about 94%, or about 95%, about 96%, about 97%, about 98%, or about 99% identical to the amino acid sequence set forth in SEQ ID NO: 12 with a sample from the subject; and
b) detecting a complex formed between the sample and the fusion protein, the composition, or the solid phase;
wherein an increased level of detected complexes relative to a normal control indicates that the subject has an E. ewingii infection.

18. The method of claim 16, wherein the antibiotic is selected from clindamycin, metronidazole, amoxicillin, cephalexin, doxycycline, enrofloxacin, gentamicin, trimethoprim, sulfamethoxazole, clavamox, chloramphenicol, cefpodoxime, tetracycline, marbofloxacin, antirobe, ciprofloxacin, albon, amoxicillin, baytril, biomox amoxicillin, cephalexin, amoxicillin, clavulanic acid, ketoconazole, sulfadimethoxine, or a combination thereof.

19. The method of claim 18, wherein the antibiotic is doxycycline.

20. The method of claim 16, wherein the subject is a dog.


SEQUENCE LISTING (25 sequences)

SEQ ID NO: 1 - protein, 20 aa, synthetic construct
AETKKTFGLEVSYDGAKIED

SEQ ID NO: 2 - protein, 20 aa, synthetic construct
AETIATFGLSKTYNGAQITD

SEQ ID NO: 3 - protein, 20 aa, synthetic construct
AETKKVLGLNKNYDGAKIED

SEQ ID NO: 4 - protein, 17 aa, synthetic construct
AETKRTFGLDKNEGLTD

SEQ ID NO: 5 - protein, 20 aa, synthetic construct
AETKNSIALEKNYDGAKIED

SEQ ID NO: 6 - protein, 20 aa, synthetic construct
AETSKKFVLENNYDGAKIED

SEQ ID NO: 7 - protein, 20 aa, synthetic construct
AETKRTFGLDIGYFGAKIED

SEQ ID NO: 8 - protein, 20 aa, synthetic construct
AETKKTFGLESFYEGAKIED

SEQ ID NO: 9 - protein, 20 aa, synthetic construct
AETKKTFGLEGSYEGAKIED

SEQ ID NO: 10 - protein, 230 aa, synthetic construct
AETKKTFGLEVSYDGAKIEDGGAETIATFGLSKTYNGAQITDGGAETKKVLGLNKNYDGAKIEDGAETKRTFGLDKNEGLTDGGAETKNSIALEKNYDGAKIEDGGAETIATFGLSKTYNGAQITDAETSKKFVLENNYDGAKIEDGGAETKRTFGLDIGYFGAKIEDGAETKKTFGLESFYEGAKIEDAETKKTFGLEGSYEGAKIEDGAETIATFGLSKTYNGAQITD

SEQ ID NO: 11 - protein, 232 aa, synthetic construct
AETKKTFGLEVSYDGAKIEDGGAETIATFGLSKTYNGAQITDGGAETKKVLGLNKNYDGAKIEDGAETKRTFGLDKNEGLTDGGAETKNSIALEKNYDGAKIEDGGAETIATFGLSKTYNGAQITDGAETSKKFVLENNYDGAKIEDGGAETKRTFGLDIGYFGAKIEDGAETKKTFGLESFYEGAKIEDGAETKKTFGLEGSYEGAKIEDGAETIATFGLSKTYNGAQITD

SEQ ID NO: 12 - protein, 236 aa, synthetic construct
SNIGAETKKTFGLEVSYDGAKIEDGGAETIATFGLSKTYNGAQITDGGAETKKVLGLNKNYDGAKIEDGAETKRTFGLDKNEGLTDGGAETKNSIALEKNYDGAKIEDGGAETIATFGLSKTYNGAQITDGAETSKKFVLENNYDGAKIEDGGAETKRTFGLDIGYFGAKIEDGAETKKTFGLESFYEGAKIEDGAETKKTFGLEGSYEGAKIEDGAETIATFGLSKTYNGAQITD

SEQ ID NO: 13 - protein, 256 aa, synthetic construct
AETKKTFGLEVSYDGAKIEDGAETKKTFGLEVSYDGAKIEDGGAETIATFGLSKTYNGAQITDGGAETKKVLGLNKNYDGAKIEDGAETKKTFGLEVSYDGAKIEDGGAETKNSIALEKNYDGAKIEDGGAETIATFGLSKTYNGAQITDGAETSKKFVLENNYDGAKIEDGGAETKRTFGLDIGYFGAKIEDGAETKKTFGLESFYEGAKIEDGAETKKTFGLEGSYEGAKIEDGAETKKTFGLEGSYEGAKIED

SEQ ID NO: 14 - protein, 251 aa, synthetic construct
AETKRTFGLDKNEGLTDGAETIATFGLSKTYNGAQITDGGAETIATFGLSKTYNGAQITDGGAETKKVLGLNKNYDGAKIEDGAETKRTFGLDKNEGLTDGGAETIATFGLSKTYNGAQITDGGAETIATFGLSKTYNGAQITDGAETIATFGLSKTYNGAQITDGGAETIATFGLSKTYNGAQITDGGAETIATFGLSKTYNGAQITDGAETKKTFGLEGSYEGAKIEDGAETIATFGLSKTYNGAQITD

SEQ ID NO: 15 - protein, 253 aa, synthetic construct
AETKKTFGLEVSYDGAKIEDGAETIATFGLEKNYDGAKIEDGGAETIATFGLSKTYNGAQITDGGAETKKVLGLNKNYDGAKIEDGAETKRTFGLDKNEGLTDGGAETKNSIALEKNYDGAKIEDGGAETIATFGLSKTYNGAQITDGAETSKKFVLENNYDGAKIEDGGAETKRTFGLDIGYFGAKIEDGAETKKTFGLESFYEGAKIEDGAETKKTFGLEGSYEGAKIEDGAETIATFGLSKTYNGAQITD

SEQ ID NO: 16 - protein, 181 aa, synthetic construct
SNIGTEDSVSAPATEDSVSAPATEDSVSAPATEDSVSAPATEDSVSAPATEDSVSAPAGGGSYNHNTGLLDLDSDILNMLYSYNHNTGLLDLDSDILNMLYSYNHNTGLLDLDSDILNMLYGGGSKEESTPEVKAEDLQPAVDSKEESTPEVKAEDLQPAVDSKEESTPEVKAEDLQPAVD

SEQ ID NO: 17 - protein, 301 aa, synthetic construct
SNIGNTTTGVFGLKQDWDGATIKDGGNTTVGVFGLKQNWDGSAISNGGNPTVALYGLKQDWNGVSAGGNTTVGVFGIEQDWDRCVISGGNPTVALYGLKQDWEGISSGGKSTVGVFGLKHDWDGSPILKGGNTTTGVFGLKQDWDGSTISGGNTTTGVFGLKQDWDGATIKDGGNPTVALYGLKQDWNGVSAGGKSTVGVFGLKHDWDGSPILKGGNTTVGVFGIEQDWDRCVISGGNTTTGVFGLKQDWDGATIKDGGNTTVGVFGLKQNWDGSAISNGGKSTVGVFGLKHDWDGSPILK

SEQ ID NO: 18 - DNA, 699 bp, synthetic construct
  source 1..699: mol_type = other DNA
gcggaaaccaagaaaacctttggtctggaagtgagctatgacggcgcgaaaatcgaagatggtggcgcggagaccattgcgaccttcggtctgagcaagacctacaacggtgcgcagatcaccgatggtggcgcggaaaccaagaaagttctgggtctgaacaaaaactatgacggtgcgaagattgaagatggcgcggagaccaaacgtacctttggtctggataagaacgaaggtctgaccgatggtggcgcggagactaaaaacagcatcgcgctggagaagaactacgatggtgcgaagatcgaggacggtggcgcggaaaccatcgcgaccttcggcctgagcaaaacctataacggtgcgcaaatcaccgacggcgcggagaccagcaagaaatttgtgctggaaaacaactatgacggtgcgaaaattgaggacggtggcgcggagaccaagcgtaccttcggcctggatatcggttactttggcgcgaaaattgaagacggtgcggagaccaagaaaaccttcggcctggaaagcttttatgagggtgcgaagatcgaggatggtgctgaaaccaagaaaaccttcggtctggaaggcagctacgagggtgcgaaaatcgaagacggcgcggaaaccattgcgacctttggtctgagcaagacctataacggcgcgcagattaccgattaa

SEQ ID NO: 19 - DNA, 711 bp, synthetic construct
  source 1..711: mol_type = other DNA
agcaacattggtgcggaaaccaagaaaacctttggtctggaagtgagctatgacggcgcgaaaatcgaagatggtggcgcggagaccattgcgaccttcggtctgagcaagacctacaacggtgcgcagatcaccgatggtggcgcggaaaccaagaaagttctgggtctgaacaaaaactatgacggtgcgaagattgaagatggcgcggagaccaaacgtacctttggtctggataagaacgaaggtctgaccgatggtggcgcggagactaaaaacagcatcgcgctggagaagaactacgatggtgcgaagatcgaggacggtggcgcggaaaccatcgcgaccttcggcctgagcaaaacctataacggtgcgcaaatcaccgacggcgcggagaccagcaagaaatttgtgctggaaaacaactatgacggtgcgaaaattgaggacggtggcgcggagaccaagcgtaccttcggcctggatatcggttactttggcgcgaaaattgaagacggtgcggagaccaagaaaaccttcggcctggaaagcttttatgagggtgcgaagatcgaggatggtgctgaaaccaagaaaaccttcggtctggaaggcagctacgagggtgcgaaaatcgaagacggcgcggaaaccattgcgacctttggtctgagcaagacctataacggcgcgcagattaccgattaa

SEQ ID NO: 20 - DNA, 771 bp, synthetic construct
  source 1..771: mol_type = other DNA
gcggagaccaagaaaacctttggcctggaagtgagctatgacggtgcgaagattgaggatggcgcggaaaccaagaaaaccttcggtctggaagtgagctacgacggcgcgaagatcgaggatggtggcgcggaaaccattgcgacctttggtctgagcaaaacctataacggcgcgcaaattaccgacggtggcgcggagaccaagaaagtgctgggtctgaacaagaactacgacggtgcgaaaatcgaagacggtgctgagaccaagaaaaccttcggcctggaagtgagctatgacggggcgaaaattgaagacggtggcgcggaaaccaaaaacagcattgcgctggaaaagaactatgatggtgcgaaaattgaagatggtggcgcggagaccattgcgaccttcggcctgagcaaaacctacaacggtgcgcaaatcaccgatggtgcggagaccagcaagaaatttgtgctggaaaacaattacgatggcgcgaagattgaggacggtggcgcggagactaaacgtaccttcggtctggacatcggttacttcggtgcgaaaattgaggatggtgctgaaaccaagaaaacctttggtctggagagcttttatgaaggtgcgaaaatcgaggacggtgccgagaccaagaaaaccttcgggctggagggcagctacgaaggcgcgaaaatcgaagacggagctgagaccaagaaaaccttcggactggagggcagctatgaaggcgcgaagatcgaagactaa

SEQ ID NO: 21 - DNA, 756 bp, synthetic construct
  source 1..756: mol_type = other DNA
gcggaaaccaagcgtacctttggcctggacaaaaacgagggtctgaccgatggcgcggaaaccattgcgacctttggtctgagcaagacctataacggtgcgcagatcaccgatggtggcgcggagaccattgcgaccttcggtctgagcaaaacctacaacggtgcgcaaattaccgatggtggcgcggaaaccaagaaagtgctgggtctgaacaagaactatgacggtgcgaaaattgaggatggcgcggagaccaaacgtaccttcggtctggacaaaaacgagggcctgaccgacggtggcgcggagaccatcgcgaccttcggcctgagcaagacctacaacggcgcgcaaatcaccgacggtggcgcggaaaccatcgcgacctttggcctgagcaaaacctataatggcgcgcagatcaccgacggtgctgagactatcgctacttttggcctgagcaagacctataatggcgcgcaaataactgatggtggcgcggagactatcgctactttcggtctgagcaagacctacaatggtgcgcaaatcactgatggtggcgcggaaactatcgctacttttggtctgagcaaaacctataacggtgcgcaaatcaccgacggcgcggagaccaagaaaacctttggtctggagggcagctatgaaggtgcgaagatcgaggacggtgctgaaaccatcgctactttcggattaagcaaaacctataacggcgcgcagattaccgactaa

SEQ ID NO: 22 - protein, 8 aa, synthetic construct
DYKDDDDK

SEQ ID NO: 23 - protein, 6 aa, synthetic construct
HHHHHH

SEQ ID NO: 24 - protein, 4 aa, synthetic construct
HHHH

SEQ ID NO: 25 - protein, 5 aa, synthetic construct
HHHHH